(19) United States
(12) Patent Application Publication
(10) Pub. No.: US 2006/0024234 A1
(43) Pub. Date: Feb. 2, 2006

(54) Protein markers for pharmaceuticals and related toxicity

(75) Inventors: N. Leigh Anderson, Washington, DC (US); Sandra C. Steiner, Gaithersburg, MD (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE PROTEOMICS CORPORATION, A Division of Large Scale Biology Corporation, Vacaville, CA (US)

(21) Appl. No.: 11/231,133

(22) Filed: Sep. 19, 2005

Related U.S. Application Data
(62) Division of application No. 09/585,475, filed on Jun. 2, 2000, now abandoned.

(51) Int. Cl. A61K 49/00

(52) U.S. Cl. 424/9.2

(57) ABSTRACT

Protein markers of toxicity and efficacy for antilipemic drugs are determined. Methods and reagents are disclosed for determining whether a patient receiving an antilipemic drug, especially a statin or HMGCoA reductase inhibiting drug, is experiencing drug efficacy and/or toxicity. Individual susceptibility is also determined prior to treatment. Also, drug discovery of similar acting candidates and their likelihood of being toxic or effective is determined by analysis of all proteins in a sample simultaneously by 2-dimensional gel electrophoresis.

### FIELD OF THE INVENTION

The present invention relates the discovery of lipid regulating drugs, and to determination of efficacy and toxicity.

### BACKGROUND OF THE INVENTION

A portion of the disclosure of this patent document contains material that is subject to copyright protection. The copyright owner has no objection to the facsimile reproduction by anyone of the patent document or patent disclosure, as it appears in the Patent and Trademark Office patent file or records, but all other copyright rights whatsoever are otherwise reserved.

High levels of low-density lipoprotein (LDL) cholesterol and low levels of high-density lipoprotein (HDL) cholesterol are both considered risk factors for coronary heart disease. In addition LDL cholesterol is involved in atherogenesis. Cholesterol is synthesized predominantly in the liver and transported to various body tissues by lipoproteins in blood plasma. Therapeutic interventions to normalize elevated plasma LDL cholesterol levels in hypercholesterolemic individuals are in widespread use.

A number of proteins are involved in lipoprotein cholesterol regulation. Considerable variation between individuals regarding such metabolism exists. For example Tangier disease results from a mutation in the gene ABC1 and causes marked low HDL-cholesterol levels. A number of polymorphisms of this gene have been noted in control subjects. HDL apolipoproteins appear to be actively transported by a pathway controlled by ABC1. ABC-1 is induced by cAMP and is a mediator in the conversion of apo AI and HDL-precursor to mature HDL. Likewise, secreted phospholipases, e.g. secretory PLA2 and endothelial lipase hydrolyze HDL phospholipids, thereby influencing HDL metabolism and function. SR-BI (Cla-1) mediates cellular uptake of cholesteryl ester from HDL. ApoAI and apoE can remove cholesterol and phospholipid as well. Cholesteryl ester transfer protein (CETP) activity and lipoprotein lipase also affect HDL by reverse cholesterol transport. CETP exchanges cholesteryl ester and triglycerides between HDL and apoB, leading to a decrease in HDL-C. Thus, an individual's distribution of proteins affects cholesterol regulation.

HMG-CoA reductase inhibitors (the best known class of which are called “statins”) have been available since 1987 and have become one of the most widely prescribed families of drugs. Statins lower LDL-C, apo B and triglycerides and raise HDL-C and apo A-I. HMG-CoA reductase is an essential regulatory enzyme in the biosynthetic pathway for cholesterol and catalyzes the conversion of HMG-CoA to mevalonate. The inhibition of this enzyme results in both the down-regulation of cholesterol synthesis and the up-regulation of hepatic high affinity receptors for low density lipoproteins (LDL) followed by increased catabolism of LDL cholesterol. Otherwise, HMG-CoA reductase inhibitors do not affect to a significant extent the levels and/or composition of the other major lipoprotein fractions. Sirtori, Pharmacological Research. 22:555-563 (1990).

Current commercially sold statin-class drugs include: lovastatin (Mevacor®), cerivastatin (Baycol®), fluvastatin (Lescol®), pravastatin sodium (Pravacol®), atorvastatin (Lipitor®) and simvastatin (Zocor®). Lovastatin and others are administered as prodrugs in their lactone forms and undergo first-pass metabolism, hepatic sequestration and hydrolysis to the beta-hydroxy acid active form. Slater et al, Drugs, 36:72-82 (1988). Thus, they appear in much higher concentrations in the liver than in non-target organs and the liver is their primary site of both, action and side effects.

Long term use of these drugs result in marked increases in serum transaminases and biochemical abnormalities of liver function in a small (≈1.9%) subset of patients who received HMG-CoA reductase inhibitors and other lipid-lowering agents. See the Physician's Desk Reference.

Toxicity testing in early drug development has changed little in decades. Toxicity is predominantly evaluated in laboratory animals using hematological, clinical chemistry and histological parameters as indicators of organ or tissue damage.

Statin drugs are known to alter the protein pattern of various cells as detectable by 2-dimensional gel electrophoresis (2DGE). Anderson et al, Electrophoresis, 12: 907-930 (1991), Gromov et al, Electrophoresis, 17(11):1728-1733 (1996), Maltese et al, Journal of Biological Chemistry 265(29):17883-17890 (1990) and Patterson et al, Journal of Biological Chemistry 270(16):9429-9436 (1995).

Other drugs are known for their antilipemic effects. Niacin and Fibric acid derivatives raise HDL, with Niacin particularly raising HDL-C while reducing LDL-C.

Other cholesterol-lowering drugs include: probucol (Lorelco®), gemfibrozil (Lopid®), niacin/nicotinic acid (Nicolar®), clofibrate (Atromid-S®), fenofibrate (Tricor®), colestipol (Colestid®) and cholestyramine (Questran®). In addition, a change in diet, particularly intake of cholesterol and fats, has an effect on the blood lipid concentration.

Most cellular proteins are post-translationally modified under normal physiological conditions. Over 200 amino acid modifications are known to occur in vivo. Krishna et al, Protein Structure—A Practical Approach, 2^{nd} ed. Creighton, ed. Oxford Univ. Press, 91-116 (1997). Given such variation, it is understandable that functional genomics has significant limitations in determining physiological changes.

Tissue proteome analysis has previously been applied to investigate the molecular effects of drugs and to obtain information on their action. Arce et al, Life Sci., 63: 2243-50 (1998), Anderson et al, Toxicol. Pathol. 1996, 24, 72-6, Anderson et al, Toxicol. Appl. Pharmacol. 1996, 137, 75-89, Steiner et al, Biochem. Biophys. Res. Commun. 1996, 218, 777-82, Aicher et al, Electrophoresis 1998, 19, 1998-2003, Myers et al, Chem. Res. Toxicol. 1995, 8, 403-13, Cunningham et al, Toxicol. Appl. Pharmacol. 1995, 131, 216-23 and Steiner et al, Biochem. Pharmacol. 51(3):253-258 (1996). Long term application of various anti-lipemic drugs is associated with hepatotoxicity in rodent studies.

Proteomics typically uses two-dimensional gel electrophoresis as a separation technique and mass spectrometry as a protein identification technique though other advanced separation and detection systems may be used.

The use of radioactive substrates to trace metabolites acted upon by various enzymes is a well-known traditional biochemical technique. Such has been used to determine enzyme activity and follow the molecule throughout metabolism and distribution in an animal.

### SUMMARY OF THE INVENTION

The object of the present invention is to determine the degree of efficacy and potential toxicity resulting from administration of an antilipemic agent by detection and/or quantification of at least one protein marker indicative of drug toxicity or efficacy in a biological sample.

It is a further object of the present invention to determine protein markers and other proteins that are potential targets for antilipemic agents and to enable screening of compounds against such proteins. Proteins strongly regulated by an antilipemic agent may serve as alternative drug targets.

It is another object of the present invention to determine other components in the metabolic pathway than the one targeted by the effective agent, toxic or therapeutic intervention by detection of at least one protein marker.

It is yet another object of the present invention to determine efficacy and toxicity protein markers for antilipemic agents and establish as protein markers themselves, both known proteins and newly discovered proteins.

It is still another object of the present invention to screen for new classes of agents having similar biological effects by detecting the effects on at least one protein marker, particularly the effects on IPP isomerase.

It is another further object of the present invention to screen for new agents that will ameliorate the effects of toxicity by detecting the effects on at least one protein marker of toxicity.

It is yet another further object of the present invention to compare protein markers of candidate drugs to protein markers for known antilipemic agents to determine comparative efficacy, toxicity and whether similar mechanisms of action are involved.

It is a still another object of the present invention to determine whether a subject will be susceptible to either the toxic and/or effective properties of a particular drug by measurement of susceptibility markers.

Other aspects of the invention include the protein markers themselves, proteomic displays containing abnormal abundances of the protein markers, and their many uses for research and monitoring patients. Also combinations of plural proteins constituting a combination marker may be used as other protein markers.

The present invention accomplishes this goal by determining which proteins are present in abnormal abundances in antilipemic agent-treated livers and deducing the mechanism of action from the perturbed metabolic pathway. Initially, all readily detectable proteins are measured; but after the markers are determined, an assay for the markers alone is sufficient. Both efficacy and toxicity determination assays may be made. In addition, monitoring of either patients on the drug or laboratory animals in drug discovery or pre-clinical testing protocols may utilize such an assay. Sets of perturbed protein markers provide a proteomic pattern or “signature” indicating relative toxicity and/or efficacy.

### DESCRIPTION OF THE PREFERRED EMBODIMENTS

The term “antilipemic agents” refer to chemicals that lower blood lipids, particularly LDL or cholesterol. These agents are useful as pharmaceuticals and include the “statin” family, HMGCoA reductase inhibitors, fibric acid derivatives, bile acid sequestrants, niacin, etc. While these antilipemic agents act by a variety of different mechanisms, the beneficial effects of drugs using these agents is well documented. These agents may be in purified form, as a natural product or extract.

The term “isolated”, when referring to a protein, means a chemical composition that is essentially free of other cellular components, particularly most other proteins. The term “purified” refers to a state where the relative concentration of a protein is significantly higher than a composition where the protein is not purified. Purity and homogeneity are typically determined using analytical techniques such as polyacrylamide gel electrophoresis or high performance liquid chromatography. Generally, a purified or isolated protein will comprise more than 80% of all macromolecular species present in the preparation. Preferably, the protein is purified to greater than 90% of all macromolecular species present. More preferably, the protein is purified to greater than 95% and most preferably the protein is purified to essential homogeneity, or wherein other macromolecular species are not significantly detected by conventional techniques.

The term “protein” is intended to also encompass derivatized molecules such as glycoproteins and lipoproteins as well as lower molecular weight polypeptides.

The term “protein marker” is a detectable “protein” which has its concentration, abundance, derivatization status, activity or other level altered in a statistically significant way when a host producing the protein marker has been exposed to an agent. Many protein markers are agent specific and all denote an amount property and use of the “protein”.

The term “agent” includes any chemical, physical, biological, electrical or radiation treatment or condition which is capable of modifying the abundance of a protein marker. Disease states and infection may also be considered an agent. Agents may also be inert or substances believed to be inert with the invention establishing the inertness such as proving pharmaceutically acceptable carriers are truly acceptable.

A “level” refers to abundance, derivatization status, protein variant presence, concentration, chemical or biological activity, which is detectable. An “altered level” refers to a change in the “level” when compared to a different sample. The “level” may be an actual measured amount of a protein but is generally a relative “level” of a protein compared to the “level” of other proteins or standards, which may be run in the same batch.

“Small molecules” are low molecular weight preferably organic molecules that are recognizable by receptors. Typically, small molecules are specific binding components for proteins.

The terms “binding component”, “ligand” or “receptor” may be any of a large number of different molecules, and the terms are used interchangeably sometimes.

The term “ligands” refers to chemical components in a sample that will specifically bind to receptors. A ligand is typically a protein or peptide but may include small molecules, particularly those acting as a hapten. For example, when detecting proteins in a sample by immunoassay, the proteins are the ligands.

The term “receptors” refers to chemical components in a reagent, which have an affinity for and are capable of binding to ligands. A receptor is typically a protein or peptide but may include small molecules. For example, an antibody molecule acts as a receptor.

The term “bind” includes any physical attachment or close association, which may be permanent or temporary. Generally, an interaction of hydrogen bonding, hydrophobic forces, van der Waals forces, etc., facilitates physical attachment between the ligand molecule of interest and the receptor. The “binding” interaction may be brief as in the situation where binding causes a chemical reaction to occur. This is typical when the binding component is an enzyme and the analyte is a substrate for the enzyme. Reactions resulting from contact between the binding component and the analyte are within the definition of binding for the purposes of the present invention. Binding is preferably specific. The binding may be reversible, particularly under different conditions.

The term “bound to” or “associated with” refers to a tight coupling of the two components mentioned. The nature of the binding may be chemical coupling through a linker moiety, physical binding or packaging such as in a macromolecular complex. Likewise all of the components of a cell are “associated with” or “bound to” the cell.

“Labels” include a large number of directly or indirectly detectable substances bound to another compound and are known per se in the immunoassay and hybridization assay fields. Examples include radioactive, fluorescent, enzyme, chemiluminescent, hapten, spin labels, a solid phase, particles, etc. Labels include indirect labels, which are detectable in the presence of another added reagent, such as a receptor bound to a biotin label and added avidin or streptavidin, labeled or subsequently labeled with labeled biotin simultaneously or later.

In situations where a chemical label is not used in an assay, alternative methods may be used such as agglutination or precipitation of the ligand/receptor complex, detecting molecular weight changes between complexed and uncomplexed ligands and receptors, optical changes to a surface (e.g., in the Biacore® device) and other changes in properties between bound and unbound ligands or receptors.

An “array” or “microarray” (depending on size) is generally a solid phase containing a plurality of different ligands or receptors immobilized thereto at predetermined locations. By contacting ligands under binding conditions to the microarray, one can determine ligand or receptor identity or at least part of the ligands' structure based on its location on the microarray. While not a single solid phase, a series of many different solid phases (or other labeling structure) each with a unique receptor immobilized thereon is considered a microarray. Each solid phase has unique detectable differences allowing one to determine the ligand or receptor immobilized thereon. An array may contain different receptors in physically separate locations even when they are not bound to a solid phase, for example a multiwelled plate.

The term “disease-related marker or portions thereof” as used herein refers to particular compounds or complexes which are found in abnormal abundances in a disease.

The term “biological sample” includes tissues, fluids, solids (preferably suspendable), extracts and fractions that contain proteins. These protein samples are from cells or fluids originating from an organism. The biological sample may be taken directly from the organism or tissue being affected or indirectly from the organism such as from serum or urine. In the present invention, the host is generally a plant or animal, preferably a mammal.

The term “proteome” is a large number of proteins expressed in a biological sample, representing the total, relevant portion or preferably all detectable proteins by a particular technique or combination of techniques. “Proteome analysis” is generally the simultaneous measurement of at least 100 proteins, generally at least a few hundred proteins, preferably over 1000 and most preferably plural thousands of detectable proteins from a sample when separated by various techniques. In the present invention, the proteome analysis involves two-dimensional gel electrophoresis. While this is the generally accepted technique for analyzing proteomes, other techniques are acceptable and may be used for the present invention if they generate large numbers of quantitatively detectable proteins. Another example is discussed in U.S. Patent Application Ser. No. 60/166,266.

The term “target” refers to any protein perturbed by a disease, developmental stage or after drug treatment. Frequently, a target refers to a drug development target that is capable of binding, or being altered by, an agent. Such drug development targets are suitable for screening candidate compounds either using direct binding assays or by observing a perturbed level, thereby indicating the candidate compound is appropriate for the next level of drug screening.

The terms “host”, “subject”, “individual”, and “tissue of interest” include both simple (viruses, unicellular organisms) and complex organisms (plants and animals) and their tissues, whether normal or abnormal, and various fractions (including subcellular fractions) of each of these.

A rate-limiting enzyme in the cholesterol synthesis pathway is HMG-CoA reductase that is competitively inhibited by the statin class of drugs. While such drugs are effective, liver cells alter their metabolism in an attempt to compensate for this disruption. Such secondary drug effects may contribute to the pharmacological action, e.g. the up-regulation of LDL receptors to remove LDL from the blood, but are often related to adverse reactions. By elucidating the biochemical pathways and networks affected upstream and particularly downstream from the blockade of HMG-CoA reductase, methods for better drug design and/or ways for compensating for toxic reactions may be found.

Other antilipemic agents may function by different mechanisms of action and their toxicity may be entirely different due to their different chemical nature. However, because the therapeutic effects themselves cause certain secondary drug effects, similarities were noted although the mechanism of action and chemical structure differs dramatically.

While applicants do not wish to be bound by any particular theory or mechanism of action, the following metabolism is believed to account for many of the effects of certain lipid lowering drugs and provide a logical basis for the present invention. For easy understanding of the present invention, certain theories of action have been presented. While the expected reactions are suggested, applicants do not wish to be bound by any implication that these are the only possible markers or that a marker may be indicative of multiple events.

It is likely that many proteins have plural functions and that the initial function found, for which the protein is usually named, may not be the true function of the protein in nature. Conversely, many genes produce multiple protein variants, differing by glycosylation, splicing, post-translational cleavage, etc. Each “protein variant” may have plural uses as well. This is clearly demonstrated by the data below where one version of a protein serves as a protein marker whereas a different version of the protein does not serve as a protein marker. The proteins may be of the same origin or encoded by different genes. An example of such is in HMG CoA synthetase or a cleavage or breakdown product thereof. As such, changes in the mRNA abundance would not necessarily reflect the marker utility of the protein. Thus, actual measurement of the protein abundance per se is needed.

Proteomics is uniquely useful in detecting and quantifying post-translational modifications. Not only does functional genomics (typically the measurement of different levels of mRNA) provide little information on RNA splicing, but also it is devoid of post-translational modification to produce protein variants. Measuring mRNA merely suggests a possible rate of synthesis not a rate or level of protein maturation and not a level of the protein per se present. Proteomics permits detection of very small chemical changes that change the peptide isoelectric point or mass, and hence the spot location on a 2-dimensional gel due to charge and mass differences. Given the large number of different post-translational modifications and their known changes as a consequence of disease or chemical/pharmacological exposure, the present invention considers changes in abundances of different protein “variants” to be equally important as overall amounts of the protein (all variants).

Various chromatographic, sedimentation, electrophoretic and other methods can fractionate protein mixtures and have been used to separate thousands of proteins. However, most proteins in a typical biological sample have not been isolated or identified, as such techniques are labor intensive, time consuming and most proteins are considered simply not to be of interest. These techniques separate the protein mixtures according to only one property and thus the separation may not be complete. To enhance purification and separation, multiple different separation techniques are used in series. However, in order to do so, each fraction from the first separation technique must separately be fractionated by a second technique.

To avoid problems with handling so many fractions, applicants used two dimensional gel electrophoresis that seamlessly merges two different techniques. The process involves subjecting the sample proteins to isoelectric focusing in a pH gradient, preferably in an elongated gel to hold the proteins in their separated state. The elongated gel is then placed on a gel sheet and subjected to denaturing SDS gel electrophoresis across the elongated gel through the gel sheet. Isoelectric focusing separates the proteins based on charge. Denaturing gel electrophoresis separates protein molecules based on the rate they pass through the gel, a measurement that corresponds to molecule size and is an indication of molecular weight. The 2-dimensional gels are prepared according to the methods in the examples. Other suitable protocols are known per se and found in several publications by the inventors and others.

If so desired, one may remove the glycosylation from proteins before 2DGE separates them. This will actually reduce the number of protein spots on the gel as some gene expression products have multiple glycosylations with each version of the product. In certain applications, this may be desirable.

Patients with high serum cholesterol, particularly those with high LDL levels compared to HDL levels may be evaluated based on levels and patterns of proteins from a biological sample. The likelihood of success and the absence of toxicity in treating the condition with an antilipemic agent may also be determined by proteome analysis of a biological sample from the patient after a short period of time on therapy, before toxicity becomes evident by gross symptoms or by increased serum transaminases and perhaps even before efficacy is confirmed by repeated blood cholesterol assays.

Therapy may also be tailored to the individual before beginning therapy by performing proteome analysis on a patient sample and comparing the protein pattern to protein patterns from a standardized normal and/or standardized patients known to respond to various antilipemic drugs and/or standardized patients who experience toxicity from statin or HMGCoA reductase inhibiting drugs.

Once a protein marker of interest has been identified, it may be produced by a number of different methods, many of which are unrelated to the manner by which it was identified.

Likewise, once protein markers are determined by proteome analysis, different assays for routine use in test animals or humans are preferred. Immunoassays and other binding assays are particularly preferred for protein marker quantification but when the marker is an enzyme, enzyme activity may be measured alone or in addition to binding assays.

The level of expression of a protein may be determined using well-known techniques such as immunofluorescence, ELISA, Western blot analysis, and similar techniques. Two-dimensional electrophoretic gels need not be used as long as the technique measures a predetermined set of protein(s) of interest. An extract for analysis of protein by any of these well-known techniques is made by conventional methods from the tissue, fluid sample, or fractions thereof. An antibody which specifically detects the selected protein, and which is conjugated to a known label, is prepared by methods known to those of skill in the art.

Any agent that produces similar changes in protein markers as demonstrated by the test antilipemic agents has potential use as a pharmaceutical. The dosages, formulations and routes of administration are readily determinable by those skilled in the art depending on the chemical structure of the agent itself. For example, the dosage employed would be sufficient to alter the protein markers' abundance to approximately the same extent as the alteration to the same marker caused by one or more known antilipemic pharmaceuticals such as those listed in the examples below.

Conventionally, to determine the effect of a compound on a cell or biological system, the compound is added and a single or few end products are measured. While such an approach is acceptable if one wishes to optimize production of a single product from the system (e.g. penicillin production from culture), this approach will not determine how a toxin affects the entire metabolism of a cell. The present invention permits one to determine global effects of a compound on the cell by measuring a protein involved in, or using a reagent containing receptors for, many or all enzymes in a metabolic pathway. One may also decipher the metabolic pathway by using plural agents to ease the process.

One need not determine an entire metabolic pathway to hypothesize at the remaining components. Furthermore, for drug development, the entire metabolic pathway need not be determined. For many uses, it is sufficient to know that a metabolic pathway's performance is reflected by the measurement of a single or relative small number of proteins in an otherwise large number of actual proteins in this metabolic pathway. Because some metabolic pathways cross other metabolic pathways, a single metabolite may be degraded or synthesized into multiple products. Therefore, it is desirable to know as much of the cellular metabolism as possible to determine global changes.

To further elicidate the metabolic pathway and effects of a drug on metabolism, the present invention also prepares an antisense compound to a previously determined protein marker and administers it to cells. When the gene and its sequence is known, the antisense compound to the gene or its mRNA may be prepared by any of the conventional techniques for preparing antisense compounds such as those of Vander Krol et al, Biotechniques 6:958+ (1988), U.S. Pat. Nos. 6,066,625, 6,063,626, 5,925,346, 5,910,444 and 5,859,342. Also, the antisense compound treated cells may be exposed to the drug or used as unexposed controls. By measuring the various proteins of the proteome, one can determine the effects of a particular drug on metabolism that has been altered by having a particular protein removed. In the situation of lipid lowering drugs, one can measure the effects on serum cholesterol of the antisense compound alone to confirm that the protein marker is a good drug target. By comparing the effects on serum cholesterol of the antisense compound and a particular drug, one can determine whether combination therapy is appropriate. Instead of measuring serum cholesterol, one may measure the levels of other proteins, particular the other protein markers, in the proteome.

Determination of differential abundances between two samples is also helpful in identifying disease specific markers, in plant and animal breeding, and in a large number of analytical and diagnostic determinations. While the emphasis of the experiments below is on finding and evaluating drugs for human use, the present invention is also useful for agricultural, horticultural, companion animals, and wild plants and animals.

In the present invention, high cholesterol diet is a proxy for a disease state as it is difficult to obtain both high and low serum cholesterol in the same population of inbred rats. Protein markers which are elevated in either the high cholesterol group or the drug treatment group but depressed in the other are particularly confirmed markers for the disorder. The same is true for other physiological conditions, particularly disease. In such a situation, protein markers from diseased and treated individuals are appropriate comparisons. More preferred are biological samples from diseased individuals taken before pharmaceutical treatment and matched samples from the same individuals after pharmaceutical treatment. Such a method is also more preferred for non-inbred populations such as those of humans.

The examples in the present invention used inbred rats of the same age to reduce genetic variability so that what is seen is the result from the agent. For some purposes, it is ideal to use the same subject to further reduce biological variability. For tests in humans, twins are especially preferred for the same reasons. Other test organisms are useful as well, as the present invention is equally applicable to plants, microorganisms, livestock and wildlife (zoos and in nature).

By knowing how an organism responds to a compound, one can develop better pesticides and genetically engineer the organism's metabolism to alter desirable traits, such as animals producing lower amounts of cholesterol in their milk, meat and eggs. Alternatively, the organism may be genetically altered to respond to various chemicals for the same or different purposes.

One can also use the present invention to alter the organism's metabolism so it responds with greater efficacy or less toxicity to a given compound. This is particularly useful for treating common diseases with chemicals that are otherwise not effective or overly toxic.

The present invention may be used as a proxy for traditional toxicity testing of new compounds for non-drug use such as cosmetics, pesticides (herbicides, fungicides, insecticides, rodenticides, antimicrobials, etc.), food and feed additives, fertilizers, agricultural and consumer products (for contact with an organism), waste effluent from industrial processes, etc.

Protein abundances and gene expression regulation following exposure to various biologically active agents is complementary to the information typically obtained by conventional tissue slide-based toxicity scoring. By comparing proteins expressed following treatment with a given agent to untreated controls, one can identify changes in the biochemical pathways via observed alterations in a protein marker or sets of markers that may be related to the agent's efficacy or toxicity. The assumption is that changes in protein abundance precede morphological changes and that these proteins are efficacy and toxicity markers that may be used alone in high throughput screening assays to test large numbers of agents.

The present invention is particularly useful in drug development in preclinical testing, proof-of-concept studies, phase I, II and III clinical testing. Even drug candidates, which have previously failed testing, may be “rescued” by proteomic analysis to stratify the patient population or to provide an indication that analogs of the drug candidate may overcome the reason for trial failure. Furthermore, enormous time and effort may be saved by avoiding animal and human testing of candidates which proteomic analysis can indicate are doomed to failure.

A method for quantitating the level of the proteins of the present invention is the abundance or ratio compared to a normal or untreated control, although other comparable methods are within the scope of this invention. The level of protein may also be determined absolutely or as a ratio compared to various components in the biological sample being tested.

While the examples in the present invention use a liver sample as the source of proteins, other tissues and body fluids may be used. Sources of proteins may be distant from the actual organ tissues being affected, such as measuring protein markers in serum even when the tissue being affected is the lung. Representative fluids include blood, serum, urine, saliva, feces, sputum, CSF, etc.

Depending on the protein sample source, different protein markers may be developed and used. Likewise, the base-line abundance of various protein markers may differ between rat, or other animal, and human sources of proteins. Homologous proteins from different species are preferred protein markers for both efficacy and toxicity.

While it is very useful to know the quantities of various protein ligands in a sample, in some situations, it may be useful to compare the sample to a standard or to measure differences in concentrations of various ligands from another sample. For example, disease specific makers may be deduced by determining which proteins are in higher or lower concentrations in a sample from diseased tissue as compared to normal tissue. The differential may be determined by using the present invention to determine the quantities in a normal and a diseased sample. The results from each experiment are compared to generate the differential results.

A particular protein level may be compared to total protein levels in the sample if a concentration control is desired. This will generate a coefficient to compare to standards so that control need not be run side by side every time. Total protein may be determined by measuring total protein being loaded on the gel, but preferably, it is compared to all other spots in the 2DE gel. Alternatively, one may compare a particular protein to a standard protein in the sample (natural internal control) or added to the sample (added internal control).

Proteomic techniques were used to study proteome changes in biological samples from antilipemic drug treated rats. The drugs were found to induce a complex pattern or “signature” of alterations in rat liver proteins, some of which were related to cholesterol synthesis but many were affecting other pathways and endpoints. This pattern is then usable for studying the biological effects of an agent or for high throughput screening of other agents for the degree of efficacy or toxicity.

Numerous changes in the proteome of liver cells exposed to antilipemic agents such as statin or other HMG-CoA inhibiting drugs were detected by the present invention. Several represent protein markers for efficacy and/or toxicity. Protein markers are also potential targets for other agents aimed at producing similar biological effects as well as targets for agents ameliorating the efficacy and/or toxicity action. Also changes in metabolism and further understanding of metabolic pathways are noted. For example, in lovastatin treatment, the markers for metabolic change include those in 1) cholesterol metabolism, 2) carbohydrate metabolism, 3) membrane trafficking, 4) cytoskeletal structure, 5) calcium homeostasis, 6) nucleotide metabolism, 7) amino acid metabolism, 8) protease inhibitors, 9) cell signaling, 10) apoptosis, 11) biotransformation, and 12) pheromone binding protein. Specific markers in each are described below. Some of these may also serve as new drug targets for biological effects relating to decreasing cholesterol synthesis or removal from blood. Additionally, they may be drug targets for ameliorating toxicity from this or another antilipemic drug and potentially from any other compound producing toxicity by the same pathway.

The protein spots affected by the treatment were identified and grouped based on cellular function and participation in biochemical and signaling pathways. Several interesting observation were made: i) The inhibition of the enzyme HMG-CoA reductase by inhibitors such as statins provoked a regulatory response associated with the strong induction of the enzymes cytosolic HMG-CoA synthase and IPP-isomerase. The fact that one enzyme is located down-stream and one is up-stream of the blockage demonstrates the liver's attempt to maintain normal cholesterol synthesis rates. ii) The liver response was not restricted to the previous therapeutically targeted pathway but involved other key enzymes regulating energy metabolism such as fructose-1,6-bisphosphatase and glucose-6-phosphate 1-dehydrogenase. iii) Several protein changes (e.g. senescence marker protein-30, serine protease inhibitor 2, protein kinase C inhibitor) indicated that high doses of statins were associated with cellular perturbations and an increase in cytosolic calcium, effects which are considered early indicators of toxicity. Many other interesting observations may be made particularly with different drug treated samples. Some of these changes may seem at least in part related to the decrease in weight gain in animals treated with high doses of lovastatin. These markers provide insights into the pathway regulation induced in response or secondary to the therapeutic action of the drug and suggest other protein targets for drug development.

Cholesterol Metabolism: Statin treatment increased the abundance of both cytosolic and mitochondrial HMG-CoA synthases, two enzymes with similar functions but encoded by different genes. Also note Ayte et al, Proc. Natl. Acad. Sci. U.S.A., 87:3874-3878 (1990). HMG-CoA synthase drives the condensation of acetyl-CoA with acetoacetyl-CoA to form HMG-CoA, which is the substrate for HMG-CoA reductase. HMG-CoA reductase is a rate-limiting enzyme of the cholesterol synthesis pathway and converts HMG-CoA to mevalonate. While cytosolic HMG-CoA synthase is not thought to be the target of the statins, it is involved in the cholesterol biosynthesis pathway, and mitochondrial HMG-CoA synthase is part of the ketone body synthesis pathway. For example, mitochondrial HMG-CoA synthase mRNA was found to be greatly increased by starvation, fat feeding and diabetes, Casals et al, Biochem. J., 283: 261-264 (1992). The strong induction of cytosolic HMG-CoA synthase following exposure to statins may represent a feedback reaction and attempt of the liver to compensate for the impaired cholesterol biosynthesis performance. The degree of its induction thus may reflect the pharmacological potency of an HMG-CoA reductase inhibitor to inhibit HMG-CoA reductase and hence serves as a marker to compare efficacy among members of the statin family of compounds and between families of chemically unrelated agents with a similar mode of action. Unequivocally, greater concentrations of statins result in a greater alteration in the abundance of many of the protein markers.

Isopentenyl-diphosphate delta-isomerase (IPP-isomerase) showed the most prominent effect following treatment with low and high doses of statins; its levels were induced about 2-fold and 24-fold respectively. This enzyme is part of the cholesterol biosynthesis pathway down-stream of HMG-CoA reductase and participates in the steps resulting in the conversion of mevalonate to farnesyl diphosphate. The strong induction of this enzyme following treatment with HMG-CoA reductase inhibitors is likely an additional attempt of the liver to maintain cholesterol synthesis rate during blockade of HMG-CoA reductase. Therefore, this protein represents a good target for drugs antagonizing this enzyme's activity. This enzyme is not previously known to be a drug target for cholesterol synthesis inhibition and therefore represents a new heretofore unknown drug target. Compounds inhibiting IPP-isomerase used in conjunction with HMG-CoA inhibitors are also suitable combinations for pharmacological use.

Precursor apolipoprotein A-I is strongly increased with statins. As with most of the apolipoproteins, apolipoprotein A-I is synthesized in the liver and then secreted into the blood. Its function involves the reverse transport of cholesterol from tissues to the liver, the site where cholesterol is metabolized and secreted. Thus, the increased synthesis of precursor apolipoprotein A-I is a likely part of the therapeutic effect of statins contributing to the net effect to decrease the amount of plasma cholesterol.

Carbohydrate Metabolism: Fructose-1,6-bisphosphatase, a key regulatory enzyme of gluconeogenesis that catalyzes the hydrolysis of fructose-1,6-bisphosphate to generate fructose-6-phosphate and inorganic phosphate, is decreased upon statin treatment. Deficiency of fructose-1,6-bisphosphatase is associated with fasting hypoglycemia and metabolic acidosis because of impaired gluconeogenesis, el-Maghrabi et al, Genomics 27: 520-5 (1995).

Glucose-6-phosphate 1-dehydrogenase, the first enzyme in the pentose phosphate pathway, is elevated by statins suggesting the up-regulation of the pentose phosphate pathway. Although the primary target of statins is cholesterol metabolism, in parallel it has major impacts on glucose metabolism, demonstrating the power of the regulatory network when central functions such as energy metabolism are affected. This effect may also be related to the treatment related decrease in weight gain in the high dose group.

Membrane Trafficking: Lovastatin induced a dose-dependent increase in annexin IV. The annexins are a group of homologous proteins that bind-membranes and aggregate vesicles in a calcium-dependent fashion and contain a binding site for calcium and phospholipid. Annexins provide a major pathway for communication between cellular membranes and their cytoplasmic environment and are implicated in membrane-related events along exocytotic and endocytotic pathways. The induction of annexin IV is likely related to the up-regulation of LDL receptor (as part of the pharmacological action of statins) and the subsequent up-regulation of the endocytosis-mediated transport of cholesterol-carrying lipoprotein into liver cells. As such, this protein is also, a drug target of compounds that up-regulate the LDL receptor and/or annexins as well as compounds that down regulate cholesterol synthesis.

Cytoskeletal Structure: The abundance of type I cytoskeletal cytokeratin 18 and of major vault protein increased upon treatment with high doses of a statin. Cytokeratin 18 is a subunit of cytokeratin filaments that are important components of the cytoskeletal structure. Major vault protein is required for normal vault structures, large ribonucleoprotein particles that may be involved in nucleo-cytoplasmic transport. The statin-mediated increase of proteins involved in cytoskeletal structure and membrane trafficking may be related to cellular stress induced by high doses. Thus, this protein primarily represents a marker for toxicity.

Calcium Homeostasis: Senescence marker protein-30 (SMP-30) is decreased in response to statin treatment. SMP-30, a cytosolic protein with decreased expression during senescent stages was recently reported to be identical to a calcium binding protein called regucaltin, Fujita et al, Mech. Ageing Dev. 10:7271-7280 (1999). SMP-30 is suggested to regulate calcium homeostasis by enhancing plasma membrane calcium-pumping activity. Its down-regulation in livers of rats treated with high doses of statins lead to the disregulation of calcium signaling and causes cellular stress. Thus, this protein primarily represents a marker for toxicity.

Nucleotide Metabolism: Adenosine is an endogenous modulator of intercellular signaling that provides homeostatic reductions in cell excitability during tissue stress and trauma. The inhibitory actions of adenosine are mediated by interactions with specific cell-surface G-protein coupled receptors regulating membrane cation flux, polarization, and the release of excitatory neurotransmitters. Adenosine kinase is the key intracellular enzyme regulating intra- and extracellular adenosine concentrations. Inhibition of adenosine kinase produces marked increases in extracellular adenosine levels that are localized to cells and tissues undergoing accelerated adenosine release, Kowaluk et al, Curr. Pharm. Des., 4:403-16 (1998). Thus the down-regulation of adenosine kinase following treatment with a statin may represents a mechanism of the liver to selectively enhance the protective actions of adenosine during stress. As such it would function primarily as a marker for toxicity.

Amino Acid Metabolism: 3-Hydroxyanthranilate 3,4-dioxygenase, an enzyme of tryptophan metabolism that catalyzes the synthesis of excitotoxin quinolinic acid (QUIN) from 3-hydroxyanthranilic acid, is decreased in livers of statin treated rats. A similar decrease is found in phenylalanine hydroxylase a key enzyme in phenylalanine metabolism. Its deficiency results in hyperphenylalaninemia, leading to severe mental retardation in the classical form of the disease, phenylketonuria, Lichter-Konecki et al, Mol. Genet. Metab. 67:308-16 (1999). It remains unclear why statin treatment is down regulating these two enzymes in liver but may be related to and a marker for indirect toxicity and/or indirect efficacy of a statin.

Protease Inhibitors: The serine protease inhibitors (serpins) are a family of proteins that function to control the action of serine proteases in many diverse physiological processes. The expression of serine protease inhibitor 2 (SPI-2) was reduced in inflammation. Treatments with high doses of lovastatin are likely to induce inflammatory processes in liver that may explain the observed decrease in SPI-2. As such these are primarily suitable markers for toxicity.

Cell Signaling: Lovastatin increased the abundances of protein kinase C inhibitor, a protein that acts as a regulator of the cell signaling process. Protein kinase C inhibitor activates tyrosine and tryptophan hydroxylases in the presence of calcium/calmodulin-dependent protein kinase II, and strongly activates protein kinase C. 23 kD morphine binding protein, a member of the phosphatidylethanolamine-binding protein (PEBP) family is increased upon treatment with lovastatin. A variety of biological roles have been described for members of this family, including lipid binding, membrane signal transduction, roles as odorant effector molecules or opioids and interaction with the cell-signaling machinery Banfield et al, Structure, 6:1245-54 (1998). The alterations in these proteins indicate that a statin affects cell signaling and provide suitable targets for drug discovery and markers of efficacy and toxicity.

Apoptosis: The protein product of a gene with the name “induced in androgen-independent prostate cells by effectors of apoptosis” was induced in the liver of statin treated animals. The induction of this gene has been shown to be apoptosis specific, Sells et al, Cell Growth Differ., 5:457-66 (1994), suggesting that toxic doses of lovastatin trigger apoptosis in liver cells of treated rats. Similar observations have been reported from in vitro experiments with lovastatin, Wang et al, Can. J. Neurol. Sci., 26:305-10 (1999). As elevation of intracellular calcium is central to apoptosis, this event is likely the consequence of the treatment-related disturbance of calcium homeostasis as reflected by the decrease in SMP-30 levels. Thus, it is primarily a good marker for toxicity of not only a statin but also any apoptosis related response to an agent or condition.

Biotransformation: N-hydroxyarylamine sulfotransferase, a liver specific enzyme involved in the biotransformation of endogenous and foreign substrates, is decreased by a statin. 3-mercaptopyruvate sulfotransferase, an enzyme involved in thiosulfate synthesis, is strongly increased by high doses of a statin. As such it may serve as a marker for either toxicity or efficacy.

Pheromone binding protein: Alpha-2u globulin is synthesized in the liver of male but not female rats, secreted into the bloodstream and excreted in the urine, Roy et al, Proc. Soc. Exp. Biol. Med., 121:894-899 (1966). It binds pheromones that are released from drying urine and affect the sexual behavior of females. There are a number of chemicals that induce a toxic syndrome in male rats referred to as alpha-2u globulin nephropathy. This organ specific toxicity is characterized by an accumulation of protein droplets in the proximal tubules. It was suggested that these droplets might be formed by the association between the chemical and the alpha-2u protein, Borghoff et al, Annu. Rev Pharmacol. Toxicol., 30:349-367 (1990). High doses of a statin strongly decrease the abundance of alpha-2u globulin in liver suggesting a down regulation of its synthesis or its increased secretion. It is likely that either this protein has an additional function or that its effect is incidental. In either situation, the protein may still serve a function as a marker for efficacy or toxicity or as a drug discovery target. Even if the effect is incidental, it remains of use as a toxicity or efficacy marker.

Peroxisome Proliferation: Proteins were previously reported to be strongly induced in the liver of rodents following treatment with peroxisome proliferators (Anderson et al, Toxicol. Appl. Pharmacol., 137:75-89 (1996)) or lovastatin (Anderson et al, Electrophoresis 12:907-930 (1991)). While developing the present invention, the previous proteins were identified as being a similar or perhaps even the homologous protein to peroxisomal enoyl hydratase-like protein. In the present examples, only a mild induction of this protein marker was observed. It may be used primarily as a marker for toxicity.

In the present invention, proteome analysis revealed quantitative alterations in a large number of hepatic proteins following treatment with lipid lowering pharmaceuticals such as lovastatin (Mevacor®). Lovastatin treatment significantly altered the abundance of 32 hepatic proteins (p<0.001). These and other marker proteins (p<0.005) are listed below. Other antilipemic agents produced similar results. That data is summarized in Table 1.

TABLE 1
Summary of All Proteins That Change at p < 0.005

| MSN | Lorelco ® | Lopid ® | Mevacor ® | Zocor ® | Lescol ® | Nicolar ® | Pravachol ® | Protein Identification |
|---|---|---|---|---|---|---|---|---|
| 18 | | ◯ | | | | | | 75 kD glucose related protein |
| 24 | | | ∘ | | | | | Calreticulin |
| 29 | | | x | ∘ | ∘ | | | Keratin type I cytoskeletal 18 |
| 34 | | | x | x | x | | | Unknown |
| 41 | | | | x | x | | | Keratin type II cytoskeletal 8 |
| 42 | | ◯ | | | | | | |
| 55 | | | x | ∘ | x | | | Senescence marker protein-30 |
| 59 | | | | | ∘ | | | |
| 66 | | | | | x | | | |
| 68 | | | ∘ | | | | | Actin gamma |
| 69 | | | | | x | | | |
| 73 | ∘ | | ∘ | x | | | | |
| 76 | | | ∘ | | ∘ | | | |
| 79 | | ◯ | x | x | x | | | Fructose-1,6-bisphosphatase |
| 83 | | | | | ∘ | | | |
| 89 | | | x | | | | | Fumarylacetoacetate hydrolase |
| 91 | | | | ∘ | x | | | Isovaleryl-CoA dehydrogenase |
| 97 | | | | x | x | | | Keratin type II cytoskeletal 8 |
| 99 | | | x | x | | | | Catechol O-methyl transferase |
| 101 | ∘ | | ∘ | | ∘ | | ∘ | Methionine adenosyltransferase |
| 103 | | | | ∘ | x | | | Senescence marker protein-30 |
| 104 | | | x | | | | | 23 kD morphine binding protein |
| 106 | ∘ | | | | | | | Catalase |
| 113 | | | x | ∘ | x | | | Adenosine kinase |
| 117 | ∘ | ◯ | | ∘ | ∘ | | | |
| 125 | | ◯ | x | x | x | | | N-hydroxyarylamine sulfotransferase |
| 126 | | | x | x | x | | | 3-Hydroxyanthranilate 3,4- |
| | | | | | | | | dioxygenase |
| 127 | | X | | | ∘ | | | |
| 128 | | | | | x | | | |
| 138 | | | ∘ | | | | | 4-Hydroxyphenylpyruvate dioxygenase |
| 139 | | X | | x | | | | |
| 143 | | | ∘ | | ∘ | | | |
| 142 | | | x | x | ∘ | | | Ketohexokinase |
| 148 | | | | x | | | | |
| 154 | | ◯ | | ∘ | | | | |
| 155 | | | | | ∘ | | | |
| 162 | | X | ∘ | x | ∘ | ◯ | | D-dopachrome tautomerase |
| 168 | | | x | | ∘ | | | Antiquitin |
| 172 | | | | ∘ | ∘ | | | Tropomysin |
| 178 | | | x | x | x | | | Aminoacylase |
| 182 | | X | x | x | x | | | Fructose-1,6-bisphosphatase |
| 191 | | ◯ | | | | | | Adenosylhomocysteinase |
| 197 | | | | ∘ | | | | |
| 200 | ∘ | | ∘ | | | | | Annexin VI |
| 203 | | | | ∘ | | | | |
| 218 | | | | ∘ | | | | |
| 227 | | X | | | | | ∘ | Fatty acid binding protein, liver |
| 229 | | | | ∘ | | | ∘ | |
| 232 | ∘ | | | | | | | |
| 237 | | | ∘ | | | | | |
| 238 | | | ∘ | | | | | |
| 252 | | | x | x | ∘ | | | Serine protease inhibitor 2 |
| 267 | | | | ∘ | | | | |
| 268 | | | | ∘ | | | | |
| 270 | | | | x | | | | |
| 282 | | | ∘ | x | | | | Pyruvate kinase, isozymes |
| 286 | ∘ | | | | | | | |
| 289 | | | | | x | | | |
| 292 | | | ∘ | | | | | |
| 297 | | | x | | | | | Unknown |
| 305 | | | x | | x | | | Phenylalanine hydroxylase |
| 307 | | | | | x | | | Heme oxygenase-1 |
| 310 | | | | | | X | | |
| 311 | | | | | ∘ | | | |
| 315 | | | ∘ | x | x | | | Pyruvate kinase L |
| 318 | | | | ∘ | x | | | |
| 321 | | | x | x | | | | Glutathione synthetase |
| 339 | ∘ | ◯ | | | | | | |
| 347 | | X | | ∘ | | | | |
| 350 | | | | ∘ | | | | |
| 358 | | | | | x | | | |
| 361 | | | x | | x | | | HMG-CoA synthase, mitochondrial |
| | | | | | | | | fragment |
| 362 | | X | ∘ | ∘ | x | ◯ | | |
| 367 | | X | | x | | | | Peroxisomal enoyl hydratase-like protein |
| 371 | | | | ∘ | ∘ | | | |
| 372 | | | x | ∘ | ∘ | | | |
| 379 | | ◯ | | | | | | |
| 384 | | | | | | ◯ | | |
| 399 | | | ∘ | | | | | |
| 413 | | X | x | x | x | | ∘ | HMG-CoA synthase, cytosolic |
| 416 | ∘ | | | | | | | |
| 420 | | X | | x | ∘ | | | |
| 427 | | ◯ | | | | | | |
| 434 | | | | | | ◯ | | |
| 435 | | | | | | ◯ | | |
| 438 | | | ∘ | | | | | |
| 457 | | | ∘ | | | | | Annexin VI |
| 463 | | | x | x | | ◯ | | Apolipoprotein A-I |
| 469 | | | | | | ◯ | | |
| 490 | | X | x | x | | | | Alpha 2u-globulin |
| 492 | | | | | ∘ | | | |
| 497 | | | ∘ | ∘ | | | | |
| 501 | | ◯ | | x | x | | | 2-oxoisovalerate dehydrogenase |
| | | | | | | | | alpha subunit, mitochondrial |
| 506 | | | | ∘ | ∘ | | | |
| 510 | | ◯ | | | | | | |
| 522 | | | ∘ | | | | | |
| 532 | | | x | | | | | Protein kinase C Inhibitor |
| 534 | | x | | | | | | ER60 protease; 58 kD microsomal protein |
| 546 | | x | | | | | | |
| 557 | | | | ∘ | | | | |
| 565 | | | | ∘ | | | | |
| 569 | x | | | | | | | |
| 590 | | x | x | x | ∘ | | | Glucose-6-phosphate 1-dehydrogenase |
| 571 | | | | ∘ | | | | |
| 574 | | | | ∘ | | | | Nucleolar phosphoprotein b23 |
| 577 | | | ∘ | | | | | Fructose-1,6-bisphophatase |
| 605 | | | | | | ◯ | | |
| 610 | | | x | | | X | | 3-Mercaptopyruvate sulfotransferase |
| 613 | | | | | ∘ | | | |
| 618 | | | ∘ | | | | | |
| 637 | | | | x | | | | |
| 644 | | | | | ∘ | | | |
| 653 | | ∘ | | | | | | |
| 664 | | | x | x | | | | Major Vault Protein |
| 665 | | | ∘ | ∘ | | | | |
| 666 | | | | | | ◯ | | |
| 669 | | | | | x | | | |
| 671 | | | ∘ | | | | | Nucleolar phosphoprotein b23 |
| 681 | | | | | x | | | |
| 689 | | | ∘ | | | | | |
| 698 | | | x | ∘ | | | | Cytokeratin ends A |
| 716 | | | | x | | | | Heterogeneous nuclear |
| | | | | | | | | ribonucleoprotein K |
| 718 | x | | | | | | | |
| 719 | | | ∘ | | | | | |
| 721 | | | | | ∘ | | | |
| 734 | | | | ∘ | | | | Lamin b |
| 777 | | | ∘ | ∘ | | | | |
| 779 | | | | x | | | | |
| 787 | | x | ∘ | | | | | |
| 802 | | | | | ∘ | | | |
| 806 | | | ∘ | | | | | |
| 810 | | | | x | | | | |
| 839 | | | ∘ | | | | | |
| 876 | | | ∘ | | | | | |
| 879 | | | | ∘ | | | | |
| 887 | | ∘ | | | | | | |
| 888 | | | ∘ | | | | | |
| 900 | | | | | ∘ | | | |
| 905 | ∘ | | | | | | | |
| 932 | | | | x | | ◯ | | Annexin IV |
| 933 | | | x | ∘ | | | | HMG-CoA synthase, cytosolic |
| 934 | | | ∘ | | ∘ | | | Ras-GTPase-activating protein |
| | | | | | | | | SH3-domain binding protein |
| 966 | | | ∘ | | | | | |
| 993 | | | x | | | | | Induced in androgen-independent |
| | | | | | | | | cells by effects of apoptosis |
| 1081 | | | x | | | | | |
| 1053 | | x | | | | | | |
| 1119 | | | x | x | x | | | Isopentyl-diphosphate delta-isomerase |
| 1250 | | | ◯ | x | ∘ | | x | HMG Co-A synthase |

x P < 0.001
∘ P < 0.005

The supporting data is presented in detail in Table 2 below.

TABLE 2
Report Data for All Significant Proteins p < 0.005
Lorelco ® Report Data for All Significant Proteins P < 0.005

| | Control | | Low Dose | | | | | High Dose | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 73 | 19379 | 0.044 | 18113 | 0.077 | 0.11911 | 0.93 | 1.07 | 16433 | 0.067 | 0.00188 | 0.85 | 1.18 |
| 101 | 13120 | 0.091 | 10860 | 0.191 | 0.06603 | 0.83 | 1.21 | 9189 | 0.141 | 0.00139 | 0.70 | 1.43 |
| 106 | 7287 | 0.149 | 6522 | 0.203 | 0.62640 | 0.89 | 1.12 | 2822 | 0.196 | 0.00106 | 0.39 | 2.58 |
| 117 | 27955 | 0.068 | 26208 | 0.078 | 0.19874 | 0.94 | 1.07 | 21878 | 0.080 | 0.00109 | 0.78 | 1.28 |
| 200 | 4725 | 0.060 | 3920 | 0.070 | 0.00219 | 0.83 | 1.21 | 3256 | 0.199 | 0.00202 | 0.69 | 1.45 |
| 232 | 4443 | 0.072 | 2706 | 0.298 | 0.00242 | 0.61 | 1.64 | 3292 | 0.155 | 0.00457 | 0.74 | 1.35 |
| 286 | 1035 | 0.171 | 633 | 0.146 | 0.00235 | 0.61 | 1.64 | 1052 | 0.196 | 0.88986 | 1.02 | 1.02 |
| 339 | 6897 | 0.049 | 4746 | 0.211 | 0.00222 | 0.69 | 1.45 | 5624 | 0.195 | 0.03689 | 0.82 | 1.23 |
| 416 | 1740 | 0.022 | 1442 | 0.514 | 0.58883 | 0.83 | 1.21 | NA | NA | NA | NA | NA |
| 569 | 3714 | 0.033 | 3222 | 0.040 | 0.00049 | 0.87 | 1.15 | 3386 | 0.043 | 0.00492 | 0.91 | 1.10 |
| 718 | 622 | 0.170 | 927 | 0.344 | 0.07451 | 1.49 | 1.49 | 1276 | 0.088 | 0.00043 | 2.05 | 2.05 |
| 905 | 709 | 0 | 945 | 0.036 | 0.00435 | 1.33 | 1.33 | 630 | 0.056 | 0.17395 | 0.89 | 1.13 |

Lorelco ® Report Data for All Significant Proteins P < 0.005

| | Control | | Low Dose | | | | | High Dose | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 18 | 29544 | 0.103 | 32303 | 0.121 | 0.24860 | 1.09 | 1.09 | 37881 | 0.090 | 0.00390 | 1.28 | 1.28 |
| 42 | 22740 | 0.116 | 16694 | 0.068 | 0.00270 | 0.73 | 1.36 | 15632 | 0.256 | 0.01850 | 0.69 | 1.45 |
| 79 | 16829 | 0.112 | 15003 | 0.121 | 0.15517 | 0.89 | 1.12 | 12088 | 0.148 | 0.00382 | 0.72 | 1.39 |
| 117 | 32744 | 0.148 | 25506 | 0.140 | 0.02658 | 0.78 | 1.28 | 19848 | 0.219 | 0.00254 | 0.61 | 1.65 |
| 125 | 26196 | 0.172 | 20200 | 0.137 | 0.03373 | 0.77 | 1.30 | 13518 | 0.215 | 0.00105 | 0.52 | 1.94 |
| 127 | 12629 | 0.025 | 14368 | 0.078 | 0.01046 | 1.14 | 1.14 | 15894 | 0.049 | 0.00011 | 1.26 | 1.26 |
| 139 | 17977 | 0.129 | 14843 | 0.141 | 0.05326 | 0.83 | 1.21 | 11200 | 0.083 | 0.00054 | 0.62 | 1.61 |
| 154 | 5146 | 0.127 | 6526 | 0.225 | 0.08881 | 1.27 | 1.27 | 6973 | 0.109 | 0.00378 | 1.36 | 1.36 |
| 162 | 28626 | 0.047 | 23899 | 0.069 | 0.00284 | 0.83 | 1.20 | 18519 | 0.112 | 0.00022 | 0.65 | 1.55 |
| 182 | 7055 | 0.054 | 6719 | 0.161 | 0.53581 | 0.95 | 1.05 | 5116 | 0.112 | 0.00045 | 0.73 | 1.38 |
| 191 | 10219 | 0.047 | 11553 | 0.085 | 0.02525 | 1.13 | 1.13 | 26789 | 0.324 | 0.00377 | 2.62 | 2.62 |
| 227 | 32925 | 0.034 | 37299 | 0.118 | 0.06135 | 1.13 | 1.13 | 57637 | 0.147 | 0.00040 | 1.75 | 1.75 |
| 339 | 8033 | 0.135 | 5651 | 0.385 | 0.05801 | 0.70 | 1.42 | 4807 | 0.213 | 0.00164 | 0.60 | 1.67 |
| 347 | 2933 | 0.068 | 2170 | 0.328 | 0.04826 | 0.74 | 1.35 | 1213 | 0.400 | 0.00022 | 0.41 | 2.42 |
| 362 | 3531 | 0.169 | 2602 | 0.262 | 0.04937 | 0.74 | 1.36 | 1614 | 0.172 | 0.00095 | 0.46 | 2.19 |
| 367 | 8319 | 0.185 | 12289 | 0.278 | 0.04359 | 1.48 | 1.48 | 20190 | 0.211 | 0.00064 | 2.43 | 2.43 |
| 379 | 5338 | 0.142 | 4582 | 0.135 | 0.14017 | 0.86 | 1.16 | 3008 | 0.270 | 0.00353 | 0.56 | 1.77 |
| 413 | 6750 | 0.659 | 17011 | 0.399 | 0.02144 | 2.52 | 2.52 | 24277 | 0.237 | 0.00095 | 3.60 | 3.60 |
| 420 | 3467 | 0.100 | 4978 | 0.121 | 0.00160 | 1.44 | 1.44 | 5816 | 0.101 | 0.00018 | 1.68 | 1.68 |
| 427 | 3374 | 0.108 | 4947 | 0.249 | 0.02450 | 1.47 | 1.47 | 5252 | 0.145 | 0.00142 | 1.56 | 1.56 |
| 490 | 9100 | 0.152 | 9218 | 0.071 | 0.86110 | 1.01 | 1.01 | 4712 | 0.227 | 0.00078 | 0.52 | 1.93 |
| 501 | 3890 | 0.106 | 3384 | 0.178 | 0.15740 | 0.87 | 1.15 | 2538 | 0.254 | 0.00447 | 0.65 | 1.53 |
| 510 | 4532 | 0.166 | 3069 | 0.391 | 0.05788 | 0.68 | 1.48 | 2248 | 0.264 | 0.00101 | 0.50 | 2.02 |
| 534 | 2189 | 0.180 | 3216 | 0.555 | 0.24385 | 1.47 | 1.47 | 4253 | 0.125 | 0.00053 | 1.94 | 1.94 |
| 546 | 1647 | 0.130 | 2479 | 0.353 | 0.07033 | 1.51 | 1.51 | 2711 | 0.096 | 0.00026 | 1.65 | 1.65 |
| 590 | 1124 | 0.278 | 3474 | 0.372 | 0.00996 | 3.09 | 3.09 | 3943 | 0.230 | 0.00095 | 3.51 | 3.51 |
| 653 | 2730 | 0.303 | 3539 | 0.213 | 0.17256 | 1.30 | 1.30 | 4864 | 0.177 | 0.00427 | 1.78 | 1.78 |
| 787 | 339 | 0.206 | 537 | 0.331 | 0.04740 | 1.58 | 1.58 | 704 | 0.172 | 0.00064 | 2.08 | 2.08 |
| 887 | 1169 | 0.306 | 1677 | 0.414 | 0.24068 | 1.43 | 1.43 | 2016 | 0.055 | 0.00450 | 1.72 | 1.72 |
| 1053 | 945 | 0.176 | | | | | | 1592 | 0.001 | 0.00422 | 1.69 | 1.69 |

Mevacor ® Report Data for All Significant Proteins p < 0.005

| | Control | | Low Dose | | | | | High Dose | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 24 | 13787 | 0.074 | 12534 | 0.272 | 0.54140 | 0.91 | 1.10 | 10058 | 0.188 | 0.00498 | 0.73 | 1.37 |
| 29 | 34250 | 0.150 | 32869 | 0.268 | 0.76613 | 0.96 | 1.04 | 66259 | 0.101 | 0.00012 | 1.93 | 1.93 |
| 34 | 18192 | 0.159 | 17176 | 0.355 | 0.74285 | 0.94 | 1.06 | 7537 | 0.093 | 0.00015 | 0.41 | 2.41 |
| 55 | 48639 | 0.091 | 54952 | 0.235 | 0.33209 | 1.13 | 1.13 | 28352 | 0.130 | 0.00016 | 0.58 | 1.72 |
| 68 | 17713 | 0.065 | 14356 | 0.144 | 0.01276 | 0.81 | 1.23 | 34457 | 0.222 | 0.00163 | 1.95 | 1.95 |
| 73 | 20326 | 0.129 | 19910 | 0.031 | 0.73694 | 0.98 | 1.02 | 13615 | 0.192 | 0.00395 | 0.67 | 1.49 |
| 76 | 11078 | 0.104 | 11516 | 0.101 | 0.57102 | 1.04 | 1.04 | 7618 | 0.136 | 0.00141 | 0.69 | 1.45 |
| 79 | 16899 | 0.046 | 14986 | 0.100 | 0.03354 | 0.89 | 1.13 | 9031 | 0.098 | 0.00002 | 0.53 | 1.87 |
| 89 | 49848 | 0.065 | 47940 | 0.086 | 0.55670 | 0.96 | 1.04 | 38872 | 0.048 | 0.00086 | 0.78 | 1.28 |
| 99 | 14417 | 0.072 | 15223 | 0.063 | 0.23788 | 1.06 | 1.06 | 20248 | 0.108 | 0.00098 | 1.40 | 1.40 |
| 101 | 13094 | 0.096 | 9480 | 0.191 | 0.00644 | 0.72 | 1.38 | 9079 | 0.194 | 0.00351 | 0.69 | 1.44 |
| 104 | 23998 | 0.082 | 26046 | 0.084 | 0.15673 | 1.09 | 1.09 | 31312 | 0.064 | 0.00066 | 1.30 | 1.30 |
| 113 | 17079 | 0.045 | 13248 | 0.254 | 0.03699 | 0.78 | 1.29 | 11991 | 0.105 | 0.00018 | 0.70 | 1.42 |
| 125 | 23926 | 0.178 | 23318 | 0.137 | 0.79983 | 0.97 | 1.03 | 12249 | 0.151 | 0.00078 | 0.51 | 1.95 |
| 126 | 10311 | 0.036 | 11284 | 0.123 | 0.16526 | 1.09 | 1.09 | 5748 | 0.254 | 0.00032 | 0.56 | 1.79 |
| 138 | 10336 | 0.176 | 9511 | 0.296 | 0.61305 | 0.92 | 1.09 | 5895 | 0.082 | 0.00105 | 0.57 | 1.75 |
| 142 | 18552 | 0.062 | 17006 | 0.296 | 0.52794 | 0.92 | 1.09 | 10762 | 0.226 | 0.00039 | 0.58 | 1.72 |
| 143 | 3659 | 0.204 | 3509 | 0.268 | 0.80326 | 0.96 | 1.04 | 1865 | 0.334 | 0.00360 | 0.51 | 1.96 |
| 162 | 25948 | 0.082 | 25862 | 0.086 | 0.95321 | 1.00 | 1.00 | 17219 | 0.115 | 0.00140 | 0.66 | 1.51 |
| 168 | 12509 | 0.094 | 12564 | 0.233 | 0.96872 | 1.00 | 1.00 | 7836 | 0.087 | 0.00018 | 0.63 | 1.60 |
| 178 | 7936 | 0.073 | 7824 | 0.080 | 0.77028 | 0.99 | 1.01 | 4970 | 0.212 | 0.00084 | 0.63 | 1.60 |
| 182 | 6585 | 0.053 | 6793 | 0.088 | 0.52414 | 1.03 | 1.03 | 3320 | 0.210 | 0.00008 | 0.50 | 1.98 |
| 200 | 4809 | 0.105 | 4155 | 0.390 | 0.58210 | 0.86 | 1.16 | 7366 | 0.140 | 0.00143 | 1.53 | 1.53 |
| 237 | 16069 | 0.048 | 14809 | 0.164 | 0.30534 | 0.92 | 1.09 | 22273 | 0.108 | 0.00128 | 1.39 | 1.39 |
| 238 | 2796 | 0.112 | 24086 | 1.212 | 0.26029 | 8.61 | 8.61 | 672 | 0.338 | 0.00317 | 0.24 | 4.16 |
| 252 | 4462 | 0.149 | 5400 | 0.177 | 0.10660 | 1.21 | 1.21 | 2359 | 0.077 | 0.00082 | 0.53 | 1.89 |
| 282 | 4376 | 0.198 | 3741 | 0.407 | 0.55500 | 0.85 | 1.17 | 2287 | 0.135 | 0.00128 | 0.52 | 1.91 |
| 292 | 5517 | 0.159 | 7475 | 0.301 | 0.10489 | 1.35 | 1.35 | 2525 | 0.338 | 0.00172 | 0.46 | 2.19 |
| 297 | 6933 | 0.051 | 6384 | 0.103 | 0.13594 | 0.92 | 1.09 | 4737 | 0.102 | 0.00014 | 0.68 | 1.46 |
| 305 | 16024 | 0.082 | 12923 | 0.235 | 0.06742 | 0.81 | 1.24 | 7542 | 0.093 | 0.00003 | 0.47 | 2.12 |
| 315 | 5089 | 0.148 | 4220 | 0.244 | 0.16418 | 0.83 | 1.21 | 3204 | 0.209 | 0.00338 | 0.63 | 1.59 |
| 321 | 3081 | 0.051 | 3075 | 0.046 | 0.94723 | 1.00 | 1.00 | 4730 | 0.090 | 0.00014 | 1.54 | 1.54 |
| 361 | 9162 | 0.389 | 7093 | 0.300 | 0.29817 | 0.77 | 1.29 | 25741 | 0.091 | 0.00027 | 2.81 | 2.81 |
| 362 | 3919 | 0.157 | 4307 | 0.250 | 0.50914 | 1.10 | 1.10 | 1900 | 0.328 | 0.00224 | 0.48 | 2.06 |
| 372 | 13614 | 0.276 | 17748 | 0.413 | 0.29510 | 1.30 | 1.30 | 62244 | 0.058 | 0.00020 | 4.57 | 4.57 |
| 399 | 5981 | 0.117 | 5007 | 0.164 | 0.07596 | 0.84 | 1.19 | 7598 | 0.041 | 0.00188 | 1.27 | 1.27 |
| 413 | 5897 | 0.142 | 8336 | 0.298 | 0.06872 | 1.41 | 1.41 | 28713 | 0.087 | 0.00001 | 4.87 | 4.87 |
| 438 | 3118 | 0.216 | 1392 | 0.424 | 0.00490 | 0.45 | 2.24 | 2475 | 0.632 | 0.52282 | 0.79 | 1.26 |
| 457 | 2616 | 0.051 | 2188 | 0.386 | 0.29344 | 0.84 | 1.20 | 3448 | 0.098 | 0.00123 | 1.32 | 1.32 |
| 463 | 3276 | 0.174 | 3116 | 0.153 | 0.64695 | 0.95 | 1.05 | 7861 | 0.165 | 0.00024 | 2.40 | 2.40 |
| 490 | 8374 | 0.290 | 10144 | 0.127 | 0.18606 | 1.21 | 1.21 | 1879 | 0.396 | 0.00072 | 0.22 | 4.46 |
| 497 | 2618 | 0.079 | 2430 | 0.243 | 0.52561 | 0.93 | 1.08 | 3614 | 0.115 | 0.00174 | 1.38 | 1.38 |
| 522 | 1873 | 0.181 | 2881 | 0.123 | 0.00371 | 1.54 | 1.54 | 2080 | 0.319 | 0.57390 | 1.11 | 1.11 |
| 532 | 2344 | 0.149 | 2780 | 0.097 | 0.05667 | 1.19 | 1.19 | 4598 | 0.144 | 0.00033 | 1.96 | 1.96 |
| 577 | 2189 | 0.157 | 2124 | 0.315 | 0.84907 | 0.97 | 1.03 | 1060 | 0.289 | 0.00175 | 0.48 | 2.07 |
| 590 | 796 | 0.106 | 985 | 0.203 | 0.08469 | 1.24 | 1.24 | 2369 | 0.166 | 0.00011 | 2.98 | 2.98 |
| 610 | 2514 | 0.204 | 2994 | 0.105 | 0.14424 | 1.19 | 1.19 | 10274 | 0.107 | 0.00004 | 4.09 | 4.09 |
| 618 | 593 | 0.097 | 803 | 0.358 | 0.14603 | 1.35 | 1.35 | 991 | 0.164 | 0.00120 | 1.67 | 1.67 |
| 664 | 1022 | 0.128 | 1131 | 0.188 | 0.62610 | 1.11 | 1.11 | 2016 | 0.093 | 0.00007 | 1.97 | 1.97 |
| 665 | 1183 | 0.150 | 1789 | 0.217 | 0.01284 | 1.51 | 1.51 | 1842 | 0.180 | 0.00471 | 1.56 | 1.56 |
| 671 | 1950 | 0.328 | 2238 | 0.190 | 0.55298 | 1.15 | 1.15 | 4397 | 0.107 | 0.00338 | 2.25 | 2.25 |
| 689 | 1276 | 0.295 | 1730 | 0.194 | 0.07713 | 1.36 | 1.36 | 2736 | 0.193 | 0.00135 | 2.14 | 2.14 |
| 698 | 2059 | 0.100 | 2054 | 0.225 | 0.98243 | 1.00 | 1.00 | 5599 | 0.107 | 0.00003 | 2.72 | 2.72 |
| 719 | 701 | 0.023 | 1062 | 0.276 | 0.04456 | 1.51 | 1.51 | 1748 | 0.175 | 0.00148 | 2.50 | 2.50 |
| 777 | 1573 | 0.193 | 1446 | 0.118 | 0.55858 | 0.92 | 1.09 | 3928 | 0.269 | 0.00171 | 2.50 | 2.50 |
| 787 | 370 | 0.153 | 318 | 0.405 | 0.55582 | 0.86 | 1.16 | 605 | 0.198 | 0.00440 | 1.63 | 1.63 |
| 806 | 826 | 0.095 | 744 | 0.353 | 0.63294 | 0.90 | 1.11 | 1817 | 0.114 | 0.00265 | 2.20 | 2.20 |
| 839 | 497 | 0.494 | 654 | 0.263 | 0.29597 | 1.32 | 1.32 | 1774 | 0.290 | 0.00313 | 3.57 | 3.57 |
| 876 | 707 | 0.206 | 833 | 0.249 | 0.29964 | 1.18 | 1.18 | 1062 | 0.068 | 0.00157 | 1.50 | 1.50 |
| 888 | 700 | 0.178 | 810 | 0.380 | 0.50874 | 1.16 | 1.16 | 368 | 0.132 | 0.00199 | 0.53 | 1.90 |
| 933 | 596 | 0.360 | 667 | 0.411 | 0.66294 | 1.12 | 1.12 | 3384 | 0.053 | 0.00001 | 5.68 | 5.68 |
| 934 | 518 | 0.371 | 920 | 0.267 | 0.06645 | 1.78 | 1.78 | 1170 | 0.131 | 0.00230 | 2.26 | 2.26 |
| 966 | 743 | 0.239 | 686 | 0.449 | 0.72679 | 0.92 | 1.08 | 1533 | 0.190 | 0.00117 | 2.06 | 2.06 |
| 993 | 618 | 0.436 | 737 | 0.362 | 0.53631 | 1.19 | 1.19 | 1709 | 0.119 | 0.00024 | 2.77 | 2.77 |
| 1081 | 362 | 0.110 | 410 | 0.628 | 0.68394 | 1.13 | 1.13 | 760 | 0.208 | 0.00090 | 2.10 | 2.10 |
| 1119 | 749 | 0.085 | 1704 | 0.374 | 0.10070 | 2.28 | 2.28 | 18114 | 0.099 | 0.00029 | 24.18 | 24.18 |
| 1250 | 1101 | 0.088 | 1490 | 0.196 | 0.07104 | 1.35 | 1.35 | 6339 | 0.311 | 0.00483 | 5.76 | 5.76 |

Zocor ® Report Data for All Significant Proteins p < 0.005

| | Control | | Low Dose | | | | | High Dose | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 29 | 36966 | 0.131 | 37735 | 0.147 | 0.81550 | 1.02 | 1.02 | 87836 | 0.252 | 0.00136 | 2.38 | 2.38 |
| 34 | 22845 | 0.062 | 23543 | 0.183 | 0.73776 | 1.03 | 1.03 | 11556 | 0.172 | 0.00006 | 0.51 | 1.98 |
| 41 | 40321 | 0.166 | 35205 | 0.161 | 0.22699 | 0.87 | 1.15 | 87826 | 0.161 | 0.00031 | 2.18 | 2.18 |
| 55 | 49112 | 0.135 | 62923 | 0.081 | 0.00624 | 1.28 | 1.28 | 32534 | 0.128 | 0.00182 | 0.66 | 1.51 |
| 73 | 24370 | 0.061 | 23721 | 0.081 | 0.57183 | 0.97 | 1.03 | 15693 | 0.156 | 0.00032 | 0.64 | 1.55 |
| 79 | 19299 | 0.087 | 18941 | 0.071 | 0.71771 | 0.98 | 1.02 | 10686 | 0.145 | 0.00012 | 0.55 | 1.81 |
| 91 | 15113 | 0.123 | 15099 | 0.137 | 0.98771 | 1.00 | 1.00 | 10873 | 0.042 | 0.00143 | 0.72 | 1.39 |
| 97 | 17730 | 0.155 | 19172 | 0.165 | 0.53062 | 1.08 | 1.08 | 39301 | 0.173 | 0.00036 | 2.22 | 2.22 |
| 99 | 15517 | 0.053 | 18695 | 0.012 | 0.00013 | 1.20 | 1.20 | 22817 | 0.070 | 0.00009 | 1.47 | 1.47 |
| 103 | 6566 | 0.296 | 6835 | 0.331 | 0.83899 | 1.04 | 1.04 | 2495 | 0.241 | 0.00242 | 0.38 | 2.63 |
| 113 | 18595 | 0.048 | 19634 | 0.081 | 0.23746 | 1.06 | 1.06 | 13417 | 0.182 | 0.00251 | 0.72 | 1.39 |
| 117 | 32173 | 0.045 | 32807 | 0.041 | 0.50125 | 1.02 | 1.02 | 20723 | 0.265 | 0.00236 | 0.64 | 1.55 |
| 125 | 26558 | 0.154 | 28556 | 0.034 | 0.31901 | 1.08 | 1.08 | 13263 | 0.054 | 0.00025 | 0.50 | 2.00 |
| 126 | 11499 | 0.039 | 12740 | 0.054 | 0.00958 | 1.11 | 1.11 | 7881 | 0.044 | 0.00002 | 0.69 | 1.46 |
| 139 | 18702 | 0.111 | 17592 | 0.346 | 0.70899 | 0.94 | 1.06 | 10022 | 0.168 | 0.00023 | 0.54 | 1.87 |
| 142 | 23893 | 0.055 | 20737 | 0.164 | 0.08626 | 0.87 | 1.15 | 12642 | 0.216 | 0.00013 | 0.53 | 1.89 |
| 148 | 6970 | 0.084 | 8984 | 0.289 | 0.17553 | 1.29 | 1.29 | 9902 | 0.073 | 0.00059 | 1.42 | 1.42 |
| 154 | 5400 | 0.044 | 5250 | 0.176 | 0.73076 | 0.97 | 1.03 | 7577 | 0.156 | 0.00408 | 1.40 | 1.40 |
| 162 | 32306 | 0.054 | 23362 | 0.427 | 0.08159 | 0.72 | 1.38 | 15200 | 0.298 | 0.00016 | 0.47 | 2.13 |
| 172 | 7945 | 0.134 | 9834 | 0.019 | 0.00468 | 1.24 | 1.24 | 11635 | 0.231 | 0.02072 | 1.46 | 1.46 |
| 178 | 8623 | 0.045 | 9093 | 0.073 | 0.20875 | 1.05 | 1.05 | 6396 | 0.089 | 0.00025 | 0.74 | 1.35 |
| 182 | 8063 | 0.076 | 7814 | 0.033 | 0.57050 | 0.97 | 1.03 | 3993 | 0.185 | 0.00008 | 0.50 | 2.02 |
| 197 | 4936 | 0.115 | 5299 | 0.123 | 0.62246 | 1.07 | 1.07 | 3303 | 0.111 | 0.00203 | 0.67 | 1.49 |
| 203 | 7736 | 0.189 | 10396 | 0.109 | 0.01213 | 1.34 | 1.34 | 2784 | 0.424 | 0.00131 | 0.36 | 2.78 |
| 218 | 2761 | 0.113 | 3574 | 0.096 | 0.00472 | 1.29 | 1.29 | 2641 | 0.349 | 0.78468 | 0.96 | 1.05 |
| 229 | 15901 | 0.056 | 14713 | 0.105 | 0.17122 | 0.93 | 1.08 | 11702 | 0.157 | 0.00209 | 0.74 | 1.36 |
| 252 | 6546 | 0.151 | 5487 | 0.118 | 0.07843 | 0.84 | 1.19 | 1322 | 0.213 | 0.00004 | 0.20 | 4.95 |
| 267 | 7209 | 0.253 | 7480 | 0.073 | 0.75561 | 1.04 | 1.04 | 11474 | 0.069 | 0.00171 | 1.59 | 1.59 |
| 268 | 7333 | 0.162 | 9538 | 0.038 | 0.00966 | 1.30 | 1.30 | 11677 | 0.131 | 0.00136 | 1.59 | 1.59 |
| 270 | 71851 | 0.099 | 60680 | 0.152 | 0.07669 | 0.84 | 1.18 | 28219 | 0.378 | 0.00036 | 0.39 | 2.55 |
| 282 | 5334 | 0.173 | 3789 | 0212 | 0.02142 | 0.71 | 1.41 | 1700 | 0.282 | 0.00017 | 0.32 | 3.14 |
| 315 | 7319 | 0.151 | 6583 | 0.122 | 0.26160 | 0.90 | 1.11 | 2870 | 0.055 | 0.00010 | 0.39 | 2.55 |
| 318 | 6434 | 0.077 | 6162 | 0.020 | 0.26521 | 0.96 | 1.04 | 3386 | 0.453 | 0.00321 | 0.53 | 1.90 |
| 321 | 3590 | 0.119 | 3516 | 0.042 | 0.72395 | 0.98 | 1.02 | 5572 | 0.110 | 0.00059 | 1.55 | 1.55 |
| 347 | 3094 | 0.118 | 3116 | 0.140 | 0.93039 | 1.01 | 1.01 | 6648 | 0.250 | 0.00193 | 2.15 | 2.15 |
| 350 | 4038 | 0.135 | 4542 | 0.137 | 0.20942 | 1.12 | 1.12 | 5378 | 0.054 | 0.00163 | 1.33 | 1.33 |
| 361 | 8846 | 0.180 | 9949 | 0.059 | 0.18212 | 1.12 | 1.12 | 24663 | 0.261 | 0.00141 | 2.79 | 2.79 |
| 362 | 4472 | 0.194 | 4858 | 0.133 | 0.54654 | 1.09 | 1.09 | 2158 | 0.257 | 0.00137 | 0.48 | 2.07 |
| 367 | 8932 | 0.137 | 9036 | 0.135 | 0.89149 | 1.01 | 1.01 | 15613 | 0.228 | 0.00442 | 1.75 | 1.75 |
| 371 | 7952 | 0.119 | 6624 | 0.128 | 0.05962 | 0.83 | 1.20 | 3473 | 0.445 | 0.00188 | 0.44 | 2.29 |
| 372 | 11370 | 0.456 | | | | | | 55197 | 0.068 | 0.00103 | 4.85 | 4.85 |
| 413 | 6479 | 0.093 | 9380 | 0.122 | 0.00138 | 1.45 | 1.45 | 35722 | 0.131 | 0.00002 | 5.51 | 5.51 |
| 420 | 4101 | 0.021 | 4112 | 0.057 | 0.91981 | 1.00 | 1.00 | 1089 | 0.454 | 0.00002 | 0.27 | 3.77 |
| 463 | 3558 | 0.113 | 3784 | 0.095 | 0.62192 | 1.06 | 1.06 | 11002 | 0.128 | 0.00004 | 3.09 | 3.09 |
| 465 | 2446 | 0.124 | 2436 | 0.184 | 0.96948 | 1.00 | 1.00 | 4724 | 0.160 | 0.00047 | 1.93 | 1.93 |
| 490 | 10398 | 0.117 | 11464 | 0.102 | 0.19462 | 1.10 | 1.10 | 2658 | 0.680 | 0.00066 | 0.26 | 3.91 |
| 497 | 2813 | 0.152 | 3237 | 0.143 | 0.16902 | 1.15 | 1.15 | 5193 | 0.248 | 0.00471 | 1.85 | 1.85 |
| 501 | 4216 | 0.120 | 4407 | 0.080 | 0.51100 | 1.05 | 1.05 | 1120 | 0.352 | 0.00005 | 0.27 | 3.76 |
| 506 | 4842 | 0.144 | 4323 | 0.107 | 0.19947 | 0.89 | 1.12 | 2905 | 0.247 | 0.00283 | 0.60 | 1.67 |
| 557 | 5175 | 0.071 | 4132 | 0.093 | 0.00267 | 0.80 | 1.25 | 4227 | 0.190 | 0.04114 | 0.82 | 1.22 |
| 565 | 1761 | 0.096 | 2281 | 0.106 | 0.00454 | 1.30 | 1.30 | 3308 | 0.552 | 0.09380 | 1.88 | 1.88 |
| 571 | 2295 | 0.079 | 2397 | 0.039 | 0.30943 | 1.04 | 1.04 | 1637 | 0.125 | 0.00191 | 0.71 | 1.40 |
| 574 | 3035 | 0.169 | 3856 | 0.082 | 0.11416 | 1.27 | 1.27 | 5426 | 0.147 | 0.00288 | 1.79 | 1.79 |
| 590 | 847 | 0.064 | 1152 | 0.105 | 0.00119 | 1.36 | 1.36 | 2509 | 0.240 | 0.00050 | 2.96 | 2.96 |
| 637 | 3144 | 0.098 | 2910 | 0.125 | 0.30663 | 0.93 | 1.08 | 1492 | 0.301 | 0.00032 | 0.47 | 2.11 |
| 664 | 1169 | 0.270 | 979 | 0.069 | 0.22455 | 0.84 | 1.19 | 2327 | 0.141 | 0.00073 | 1.99 | 1.99 |
| 665 | 1549 | 0.122 | 2036 | 0.092 | 0.00376 | 1.31 | 1.31 | 2390 | 0.176 | 0.00384 | 1.54 | 1.54 |
| 698 | 2992 | 0.145 | 2347 | 0.399 | 0.19740 | 0.78 | 1.28 | 4469 | 0.022 | 0.00183 | 1.49 | 1.49 |
| 716 | 1696 | 0.212 | 2183 | 0.168 | 0.06492 | 1.29 | 1.29 | 4073 | 0.213 | 0.00074 | 2.40 | 2.40 |
| 734 | 577 | 0.326 | 687 | 0.177 | 0.30471 | 1.19 | 1.19 | 1075 | 0.132 | 0.00363 | 1.86 | 1.86 |
| 777 | 2190 | 0.088 | 1980 | 0.088 | 0.10434 | 0.90 | 1.11 | 4458 | 0.214 | 0.00112 | 2.04 | 2.04 |
| 779 | 274 | 0.076 | 338 | 0.667 | 0.72422 | 1.23 | 1.23 | 768 | 0.030 | 0.00020 | 2.80 | 2.80 |
| 810 | 547 | 0.073 | 267 | 0.208 | 0.00018 | 0.49 | 2.05 | 542 | 0.830 | 0.97731 | 0.99 | 1.01 |
| 879 | 1027 | 0.272 | 1097 | 0.226 | 0.68842 | 1.07 | 1.07 | 1980 | 0.150 | 0.00112 | 1.93 | 1.93 |
| 932 | 1062 | 0.171 | 1107 | 0.166 | 0.75740 | 1.04 | 1.04 | 3053 | 0.071 | 0.00030 | 2.87 | 2.87 |
| 933 | 340 | 0.415 | 615 | 0.174 | 0.01938 | 1.81 | 1.81 | 4179 | 0.249 | 0.00128 | 12.29 | 12.29 |
| 1119 | 1032 | 0.347 | 1654 | 0.278 | 0.09225 | 1.60 | 1.60 | 20698 | 0.220 | 0.00071 | 20.05 | 20.05 |
| 1250 | 617 | 0.469 | 1163 | 0.336 | 0.06444 | 1.88 | 1.88 | 6518 | 0.302 | 0.00095 | 10.56 | 10.56 |

Lescol ® Report Data for All Significant Proteins p < 0.005

| | Control | | Low Dose | | | | | High Dose | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 29 | 31559 | 0.535 | 36444 | 0.125 | 0.60026 | 1.15 | 1.15 | 83438 | 0.199 | 0.00282 | 2.64 | 2.64 |
| 34 | 20312 | 0.127 | 22718 | 0.105 | 0.18906 | 1.12 | 1.12 | 9706 | 0.087 | 0.00054 | 0.48 | 2.09 |
| 41 | 32634 | 0.165 | 36960 | 0.270 | 0.53081 | 1.13 | 1.13 | 88598 | 0.182 | 0.00058 | 2.71 | 2.71 |
| 55 | 56109 | 0.177 | 45205 | 0.374 | 0.29408 | 0.81 | 1.24 | 19397 | 0.132 | 0.00025 | 0.35 | 2.89 |
| 59 | 11081 | 0.152 | 13688 | 0.288 | 0.26916 | 1.24 | 1.24 | 4005 | 0.127 | 0.00162 | 0.36 | 2.77 |
| 66 | 9324 | 0.095 | 8590 | 0.230 | 0.52217 | 0.92 | 1.09 | 3231 | 0.266 | 0.00010 | 0.35 | 2.89 |
| 69 | 13513 | 0.070 | 13093 | 0.081 | 0.56095 | 0.97 | 1.03 | 8113 | 0.138 | 0.00031 | 0.60 | 1.67 |
| 76 | 12156 | 0.051 | 11697 | 0.184 | 0.69539 | 0.96 | 1.04 | 7830 | 0.198 | 0.00165 | 0.64 | 1.55 |
| 79 | 18071 | 0.069 | 17282 | 0.112 | 0.51039 | 0.96 | 1.05 | 6954 | 0.152 | 0.00004 | 0.38 | 2.60 |
| 83 | 17145 | 0.048 | 15809 | 0.246 | 0.53110 | 0.92 | 1.08 | 13222 | 0.113 | 0.00263 | 0.77 | 1.30 |
| 91 | 13293 | 0.068 | 14749 | 0.344 | 0.59789 | 1.11 | 1.11 | 8686 | 0.140 | 0.00071 | 0.65 | 1.53 |
| 97 | 14505 | 0.100 | 14542 | 0.310 | 0.98520 | 1.00 | 1.00 | 40615 | 0.113 | 0.00009 | 2.80 | 2.80 |
| 101 | 15597 | 0.122 | 13698 | 0.199 | 0.27795 | 0.88 | 1.14 | 7581 | 0.373 | 0.00231 | 0.49 | 2.06 |
| 103 | 5942 | 0.130 | 10472 | 0.076 | 0.00046 | 1.76 | 1.76 | 1504 | 0.246 | 0.00022 | 0.25 | 3.95 |
| 113 | 17110 | 0.023 | 15940 | 0.135 | 0.32555 | 0.93 | 1.07 | 11325 | 0.042 | 0.00002 | 0.66 | 1.51 |
| 117 | 27421 | 0.049 | 27482 | 0.137 | 0.97825 | 1.00 | 1.00 | 18511 | 0.115 | 0.00111 | 0.68 | 1.48 |
| 125 | 26925 | 0.086 | 27884 | 0.134 | 0.67135 | 1.04 | 1.04 | 11676 | 0.220 | 0.00016 | 0.43 | 2.31 |
| 126 | 10805 | 0.103 | 9737 | 0.320 | 0.54386 | 0.90 | 1.11 | 4289 | 0.128 | 0.00007 | 0.40 | 2.52 |
| 127 | 12234 | 0.030 | 10658 | 0.084 | 0.01370 | 0.87 | 1.15 | 9529 | 0.099 | 0.00142 | 0.78 | 1.28 |
| 128 | 3171 | 0.161 | 3360 | 0.438 | 0.81010 | 1.06 | 1.06 | 722 | 0.457 | 0.00019 | 0.23 | 4.39 |
| 142 | 20616 | 0.186 | 19066 | 0.243 | 0.61259 | 0.92 | 1.08 | 9922 | 0.202 | 0.00133 | 0.48 | 2.08 |
| 143 | 3672 | 0.117 | 3767 | 0.229 | 0.84114 | 1.03 | 1.03 | 2123 | 0.193 | 0.00126 | 0.58 | 1.73 |
| 155 | 5402 | 0.298 | 4970 | 0.210 | 0.64327 | 0.92 | 1.09 | 2038 | 0.258 | 0.00335 | 0.38 | 2.65 |
| 162 | 26401 | 0.210 | 20693 | 0.310 | 0.20151 | 0.78 | 1.28 | 12223 | 0.349 | 0.00372 | 0.46 | 2.16 |
| 168 | 12442 | 0.073 | 11582 | 0.095 | 0.30247 | 0.93 | 1.07 | 7607 | 0.154 | 0.00138 | 0.61 | 1.64 |
| 172 | 7470 | 0.100 | 7087 | 0.240 | 0.69158 | 0.95 | 1.05 | 10203 | 0.097 | 0.00297 | 1.37 | 1.37 |
| 178 | 7860 | 0.040 | 8511 | 0.082 | 0.12793 | 1.08 | 1.08 | 5012 | 0.096 | 0.00011 | 0.64 | 1.57 |
| 182 | 7344 | 0.084 | 6691 | 0.276 | 0.52915 | 0.91 | 1.10 | 1912 | 0.198 | 0.00003 | 0.26 | 3.84 |
| 252 | 4765 | 0.086 | 3799 | 0.313 | 0.16733 | 0.80 | 1.25 | 2106 | 0.317 | 0.00189 | 0.44 | 2.26 |
| 289 | 6652 | 0.082 | 5959 | 0.260 | 0.56984 | 0.90 | 1.12 | 3220 | 0.263 | 0.00045 | 0.48 | 2.07 |
| 305 | 17349 | 0.115 | 15895 | 0.121 | 0.33644 | 0.92 | 1.09 | 6888 | 0.353 | 0.00047 | 0.40 | 2.52 |
| 307 | 8061 | 0.086 | 7989 | 0.172 | 0.92413 | 0.99 | 1.01 | 4594 | 0.175 | 0.00050 | 0.57 | 1.75 |
| 311 | 9496 | 0.558 | 12808 | 0.133 | 0.22281 | 1.35 | 1.35 | 25505 | 0.145 | 0.00143 | 2.69 | 2.69 |
| 315 | 7783 | 0.082 | 7705 | 0.190 | 0.92215 | 0.99 | 1.01 | 3518 | 0.273 | 0.00033 | 0.45 | 2.21 |
| 318 | 5957 | 0.078 | 5399 | 0.141 | 0.24068 | 0.91 | 1.10 | 2803 | 0.284 | 0.00046 | 0.47 | 2.13 |
| 358 | 2923 | 0.066 | 2767 | 0.182 | 0.58426 | 0.95 | 1.06 | 1875 | 0.138 | 0.00053 | 0.64 | 1.56 |
| 361 | 6854 | 0.517 | 8516 | 0.192 | 0.62007 | 1.24 | 1.24 | 26470 | 0.127 | 0.00021 | 3.86 | 3.86 |
| 362 | 4646 | 0.081 | 5363 | 0.212 | 0.27184 | 1.15 | 1.15 | 1744 | 0.187 | 0.00016 | 0.38 | 2.66 |
| 371 | 6761 | 0.157 | 6377 | 0.130 | 0.58967 | 0.94 | 1.06 | 2989 | 0.381 | 0.00405 | 0.44 | 2.26 |
| 372 | 9972 | 0.292 | 10826 | 0.059 | 0.64733 | 1.09 | 1.09 | 62963 | 0.189 | 0.00130 | 6.31 | 6.31 |
| 413 | 5733 | 0.076 | 8971 | 0.105 | 0.00070 | 1.56 | 1.56 | 30546 | 0.185 | 0.00020 | 5.33 | 5.33 |
| 420 | 3710 | 0.080 | 3920 | 0.109 | 0.56193 | 1.06 | 1.06 | 1190 | 0.391 | 0.00219 | 0.32 | 3.12 |
| 492 | 3119 | 0.168 | 2840 | 0.260 | 0.55043 | 0.91 | 1.10 | 1534 | 0.245 | 0.00150 | 0.49 | 2.03 |
| 501 | 4382 | 0.089 | 4489 | 0.093 | 0.70636 | 1.02 | 1.02 | 930 | 0.364 | 0.00011 | 0.21 | 4.71 |
| 506 | 4425 | 0.138 | 4519 | 0.098 | 0.80571 | 1.02 | 1.02 | 2724 | 0.121 | 0.00140 | 0.62 | 1.62 |
| 590 | 948 | 0.083 | 1310 | 0.189 | 0.02653 | 1.38 | 1.38 | 2444 | 0.209 | 0.00106 | 2.58 | 2.58 |
| 613 | 1278 | 0.104 | 1350 | 0.118 | 0.51648 | 1.06 | 1.06 | 756 | 0.195 | 0.00243 | 0.59 | 1.69 |
| 644 | 2843 | 0.102 | 2351 | 0.258 | 0.18080 | 0.83 | 1.21 | 1214 | 0.492 | 0.00205 | 0.43 | 2.34 |
| 669 | 1440 | 0.072 | 1239 | 0.267 | 0.28610 | 0.86 | 1.16 | 684 | 0.184 | 0.00013 | 0.48 | 2.11 |
| 681 | 4397 | 0.168 | 3873 | 0.289 | 0.54584 | 0.88 | 1.14 | 1248 | 0.495 | 0.00045 | 0.28 | 3.52 |
| 721 | 478 | 0.102 | 209 | 0.207 | 0.00351 | 0.44 | 2.28 | | | | | |
| 802 | 1257 | 0.114 | 1163 | 0.198 | 0.51806 | 0.93 | 1.08 | 742 | 0.173 | 0.00223 | 0.59 | 1.70 |
| 900 | 1104 | 0.011 | 451 | 0.231 | 0.00220 | 0.41 | 2.45 | 851 | 0.481 | 0.54071 | 0.77 | 1.30 |
| 934 | 809 | 0.144 | 473 | 0.188 | 0.00422 | 0.58 | 1.71 | 418 | 0.544 | 0.01698 | 0.52 | 1.93 |
| 1119 | 633 | 0.093 | 1448 | 0.436 | 0.14420 | 2.29 | 2.29 | 15428 | 0.062 | 0.00013 | 24.38 | 24.38 |
| 1250 | 686 | 0.235 | 1731 | 0.278 | 0.00483 | 2.52 | 2.52 | 2175 | 0.273 | 0.00512 | 3.17 | 3.17 |

Nicolar ® Report Data for All Significant Proteins p < 0.005

| | Control | | Low Dose | | | | | High Dose | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 162 | 29115 | 0.113 | 25667 | 0.240 | 0.55675 | 0.88 | 1.13 | 17066 | 0.185 | 0.00263 | 0.59 | 1.71 |
| 310 | 15450 | 0.140 | 8840 | 0.167 | 0.00076 | 0.57 | 1.75 | 13282 | 0.081 | 0.07741 | 0.86 | 1.16 |
| 362 | 4104 | 0.156 | 3760 | 0.262 | 0.53693 | 0.92 | 1.09 | 2152 | 0.257 | 0.00119 | 0.52 | 1.91 |
| 384 | 15154 | 0.069 | 11721 | 0.187 | 0.08961 | 0.77 | 1.29 | 10637 | 0.069 | 0.00496 | 0.70 | 1.42 |
| 434 | 5799 | 0.157 | 2680 | 0.057 | 0.00173 | 0.46 | 2.16 | 4063 | 0.387 | 0.08730 | 0.70 | 1.43 |
| 435 | 7397 | 0.114 | 5828 | 0.047 | 0.00439 | 0.79 | 1.27 | 6204 | 0.165 | 0.07673 | 0.84 | 1.19 |
| 463 | 3801 | 0.149 | 3430 | 0.258 | 0.50642 | 0.90 | 1.11 | 2538 | 0.094 | 0.00212 | 0.67 | 1.50 |
| 469 | 4570 | 0.056 | 3238 | 0.319 | 0.02743 | 0.71 | 1.41 | 3823 | 0.056 | 0.00140 | 0.84 | 1.20 |
| 605 | 166 | 0.209 | 301 | 0.473 | 0.07483 | 1.81 | 1.81 | 317 | 0.244 | 0.00444 | 1.90 | 1.90 |
| 610 | 3390 | 0.079 | 2300 | 0.140 | 0.00067 | 0.68 | 1.47 | 3096 | 0.167 | 0.29242 | 0.91 | 1.09 |
| 666 | 114 | 0.127 | 190 | 0.013 | 0.00187 | 1.67 | 1.67 | 233 | 0.369 | 0.13888 | 2.05 | 2.05 |
| 932 | 2440 | 0.110 | 740 | 0.189 | 0.00154 | 0.30 | 3.30 | 1548 | 0.422 | 0.06855 | 0.63 | 1.58 |

Pravachol ® Report Data for All Significant Proteins p < 0.005

| | Control | | Low Dose | | | | | High Dose | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 101 | 9912 | 0.157 | 13615 | 0.104 | 0.00460 | 1.37 | 1.37 | 10529 | 0.240 | 0.65666 | 1.06 | 1.06 |
| 227 | 28834 | 0.147 | 19783 | 0.087 | 0.00257 | 0.69 | 1.46 | 27074 | 0.262 | 0.64946 | 0.94 | 1.07 |
| 229 | 15082 | 0.057 | 13725 | 0.071 | 0.04644 | 0.91 | 1.10 | 10693 | 0.174 | 0.00173 | 0.71 | 1.41 |
| 413 | 4951 | 0.335 | 6944 | 0.149 | 0.06015 | 1.40 | 1.40 | 14671 | 0.147 | 0.00171 | 2.96 | 2.96 |
| 1250 | 547 | 0.523 | 672 | 0.435 | 0.62727 | 1.23 | 1.23 | 2238 | 0.257 | 0.00062 | 4.10 | 4.10 |

In the present invention, a probability value of p<0.001 in a Student's t-test is generally accepted as indicating high statistical significance. While higher p values of <0.01 may be considered statistically acceptable to some, other experiments have shown that this is not acceptable for 2-dimensional gel electrophoresis with the number of samples (n=5) analyzed per group considering the variation even between inbred animals. Applicants have found from other studies that such levels of certainty are not certain at all and produce results with a high rate of false positives.

By raising the p value from <0.001 to <0.005 many other markers are selected. While one is less certain that the effects are not random artifacts, significant information regarding the metabolic and toxic pathways and other potential drug targets may be gleaned. By discovering consistency of protein markers between similar agents, the statistical significance of the marker for the class of agents increases greatly. For example lovastatin, simvastatin and fluvastatin are chemically and pharmacologically similar and with respect to keratin type I cytoskeletal 18 in the high dose, the p values are 0.0010, 0.00131, and 0.00282 respectively. Considering that the protein is a marker for lovastatin, it is logical to consider it to be a protein marker for simvastatin and fluvastatin even though the p value for each may not be considered highly significant by being above the most stringent cut-off value. Likewise, with respect to protein MSN 73, the p value for lovastatin high dose is <0.00412 and for simvastatin high dose it is <0.00025. Indeed, chemically different but also an antilipemic agent, probucol high dose has a p value of 0.00144 with respect to MSN 73. Likewise, fatty acid binding protein, liver has p values for pravastatin sodium of 0.00260 and gemfibrozil of 0.00013, even though these compounds are chemically quite different and believed to have very different modes of action. Numerous other examples are present and may be so determined. Thus, cut-offs values are arbitrary and may not accurately reflect true pharmacological and toxicological action. The markers for various drugs are given in the Tables.

In another study using an intermediate dosage and different aged rats, lovastatin, cholestyramine, high cholesterol diet and a combination of lovastatin and cholestyramine were used. The results are summarized in Table 3 below. The altered abundance of proteins compared to a control with given p values and other statistical data are given in Table 4 below. Because the experimental conditions were slightly different from the above experiment, some differences were noted; however, many markers of particularly interest are the same.

TABLE 3
Summary of All Proteins Changed
All Proteins that change at P < 0.005

| | High | | | Cholestyramine + | |
|---|---|---|---|---|---|
| MSN | Cholesterol | Cholestyramine | Lovastatin | Lovastatin | Protein Identification |
| 34 | | | | ◯ | Unknown |
| 97 | | | x | X | Keratin type II cytoskeletal 8 |
| 99 | x | | | | Catechol O-methyl transferase |
| 104 | x | | | | 23 kD morphine binding protein |
| 115 | ∘ | | | | Apolipoprotein E precursor |
| 122 | ∘ | | | | |
| 142 | | | ∘ | | Ketohexokinase |
| 147 | | | ∘ | | HumorF06 |
| 178 | | | ∘ | ◯ | Antiquitin |
| 182 | | | | ◯ | Fructose-1,6-bisphosphatase |
| 191 | | | ∘ | | Adenosylhomocysteinase |
| 204 | | | x | | Alanine aminotransferase |
| 232 | ∘ | | ∘ | | |
| 275 | ∘ | | | | |
| 279 | | | ∘ | | |
| 322 | ∘ | | | | |
| 361 | | ∘ | | | HMG -CoA synthase, mitochondrial |
| 365 | | x | | | |
| 367 | | | x | X | Peroxisomal enoyl hydratase-like protein |
| 395 | | | | ◯ | |
| 413 | | | x | ◯ | HMG -CoA synthase, cytosolic |
| 423 | | | ∘ | | |
| 461 | x | | | | |
| 475 | ∘ | | | | N-G,N-G-dimethylarginine dimethylaminohydrolase |
| 479 | | | | X | |
| 490 | ∘ | | | | Alpha 2u-globulin |
| 502 | | ∘ | | | |
| 556 | ∘ | | | | |
| 578 | | | | ◯ | |
| 590 | ∘ | | | | Glucose-6-phosphate 1-dehydrogenase |
| 602 | | ∘ | | | |
| 610 | | | ∘ | | 3-Mercaptopyruvate sulfotransferase |
| 625 | ∘ | | | | |
| 633 | | | x | | |
| 646 | x | | | | |
| 664 | ∘ | | | | Major Vault Protein |
| 984 | x | | | | |
| 998 | | | ∘ | | Epoxide hydrolase, soluble |
| 1001 | | | ∘ | ◯ | |
| 1065 | | ∘ | ∘ | | |
| 1081 | | | | ◯ | |
| 1172 | | | | ◯ | |
| 1195 | | | | ◯ | |
| 1215 | | ∘ | | | 90 KD heat shock protein |

x P < 0.001
∘ P < 0.005

TABLE 4
Report Data for All Significant Proteins
All Groups with protein significant P < 0.005 in
at least one group

| | Control | | High Cholesterol | | | | | Cholestyramine | | | | |
|---|---|---|---|---|---|---|---|---|---|---|---|---|
| MSN | AVOL | CV | AVOL | CV | PROB | RATIO | NFOLD | AVOL | CV | PROB | RATIO | NFOLD |
| 34 | 29657 | 0.079 | 32431 | 0.059 | 0.07314 | 1.09 | 1.09 | 26366 | 0.086 | 0.05268 | 0.89 | 1.12 |
| 97 | 13102 | 0.033 | 15494 | 0.184 | 0.09809 | 1.18 | 1.18 | 13914 | 0.045 | 0.04154 | 1.06 | 1.06 |
| 99 | 25928 | 0.088 | 16062 | 0.142 | 0.00031 | 0.62 | 1.61 | 24066 | 0.149 | 0.64205 | 0.93 | 1.08 |
| 104 | 28850 | 0.080 | 21958 | 0.060 | 0.00066 | 0.76 | 1.31 | 30956 | 0.057 | 0.14172 | 1.07 | 1.07 |
| 115 | 17488 | 0.118 | 22656 | 0.062 | 0.00203 | 1.30 | 1.30 | 18533 | 0.156 | 0.53363 | 1.06 | 1.06 |
| 122 | 22469 | 0.138 | 31010 | 0.094 | 0.00236 | 1.38 | 1.38 | 23536 | 0.175 | 0.65862 | 1.05 | 1.05 |
| 142 | 27870 | 0.046 | 28701 | 0.072 | 0.52574 | 1.03 | 1.03 | 28969 | 0.048 | 0.22843 | 1.04 | 1.04 |
| 147 | 27000 | 0.058 | 26211 | 0.078 | 0.51975 | 0.97 | 1.03 | 26408 | 0.044 | 0.52381 | 0.98 | 1.02 |
| 178 | 9747 | 0.044 | 9850 | 0.105 | 0.83555 | 1.01 | 1.01 | 9135 | 0.044 | 0.04680 | 0.94 | 1.07 |
| 182 | 7642 | 0.087 | 9072 | 0.116 | 0.03194 | 1.19 | 1.19 | 6264 | 0.146 | 0.02506 | 0.82 | 1.22 |
| 191 | 10964 | 0.074 | 12551 | 0.135 | 0.09301 | 1.14 | 1.14 | 9039 | 0.400 | 0.27835 | 0.82 | 1.21 |
| 204 | 6268 | 0.144 | 6752 | 0.185 | 0.52367 | 1.08 | 1.08 | 4600 | 0.291 | 0.05855 | 0.73 | 1.36 |
| 232 | 8495 | 0.214 | 4900 | 0.177 | 0.00425 | 0.58 | 1.73 | 7091 | 0.350 | 0.33863 | 0.83 | 1.20 |
| 275 | 4715 | 0.080 | 7031 | 0.083 | 0.00136 | 1.49 | 1.49 | 4049 | 0.016 | 0.10043 | 0.86 | 1.16 |
| 279 | 4852 | 0.040 | 5492 | 0.179 | 0.19038 | 1.13 | 1.13 | 4667 | 0.163 | 0.61486 | 0.96 | 1.04 |
| 322 | 13385 | 0.150 | 21180 | 0.157 | 0.00491 | 1.58 | 1.58 | 18949 | 0.355 | 0.16766 | 1.42 | 1.42 |
| 361 | 5331 | 0.167 | 4356 | 0.194 | 0.20260 | 0.82 | 1.22 | 7535 | 0.069 | 0.00271 | 1.41 | 1.41 |
| 365 | 3264 | 0.045 | 3450 | 0.117 | 0.63566 | 1.06 | 1.06 | 2721 | 0.053 | 0.00063 | 0.83 | 1.20 |
| 367 | 11735 | 0.114 | 11797 | 0.114 | 0.94212 | 1.01 | 1.01 | 14197 | 0.050 | 0.00687 | 1.21 | 1.21 |
| 395 | 5406 | 0.116 | 4998 | 0.181 | 0.56493 | 0.92 | 1.08 | 5014 | 0.149 | 0.60122 | 0.93 | 1.08 |
| 413 | 2983 | 0.157 | | | | | | 4033 | 0.275 | 0.08444 | 1.35 | 1.35 |
| 423 | 4320 | 0.100 | 5199 | 0.071 | 0.00870 | 1.20 | 1.20 | 3836 | 0.159 | 0.18263 | 0.89 | 1.13 |
| 461 | 3606 | 0.072 | 2695 | 0.074 | 0.00089 | 0.75 | 1.34 | 3919 | 0.101 | 0.21629 | 1.09 | 1.09 |
| 475 | 4565 | 0.135 | 6426 | 0.099 | 0.00187 | 1.41 | 1.41 | 4646 | 0.097 | 0.81176 | 1.02 | 1.02 |
| 479 | 2492 | 0.199 | 1909 | 0.216 | 0.07521 | 0.77 | 1.31 | 3389 | 0.205 | 0.04500 | 1.36 | 1.36 |
| 490 | 5649 | 0.052 | 3459 | 0.201 | 0.00283 | 0.61 | 1.63 | 5334 | 0.380 | 0.79884 | 0.94 | 1.06 |
| 502 | 3386 | 0.068 | 3323 | 0.164 | 0.81281 | 0.98 | 1.02 | 2628 | 0.117 | 0.00254 | 0.78 | 1.29 |
| 556 | 3004 | 0.167 | 1760 | 0.144 | 0.00145 | 0.59 | 1.71 | 3232 | 0.106 | 0.57177 | 1.08 | 1.08 |
| 578 | 2677 | 0.141 | 3116 | 0.095 | 0.07271 | 1.16 | 1.16 | 2480 | 0.203 | 0.50828 | 0.93 | 1.08 |
| 590 | 3745 | 0.262 | 1951 | 0.158 | 0.00483 | 0.52 | 1.92 | 4178 | 0.271 | 0.54203 | 1.12 | 1.12 |
| 602 | 3147 | 0.090 | 3275 | 0.107 | 0.54745 | 1.04 | 1.04 | 2492 | 0.079 | 0.00316 | 0.79 | 1.26 |
| 610 | 3055 | 0.250 | 2461 | 0.064 | 0.12544 | 0.81 | 1.24 | 3894 | 0.122 | 0.06894 | 1.27 | 1.27 |
| 625 | 3493 | 0.233 | 1994 | 0.121 | 0.00449 | 0.57 | 1.75 | 3357 | 0.159 | 0.75935 | 0.96 | 1.04 |
| 633 | 2750 | 0.102 | 3339 | 0.229 | 0.14178 | 1.21 | 1.21 | 3286 | 0.205 | 0.13761 | 1.19 | 1.19 |
| 646 | 2957 | 0.086 | 4174 | 0.046 | 0.00023 | 1.41 | 1.41 | 2977 | 0.166 | 0.94369 | 1.01 | 1.01 |
| 664 | 1115 | 0.091 | 1396 | 0.066 | 0.00212 | 1.25 | 1.25 | 954 | 0.079 | 0.03308 | 0.86 | 1.17 |
| 984 | 594 | 0.078 | 882 | 0.065 | 0.00025 | 1.48 | 1.48 | 665 | 0.126 | 0.17516 | 1.12 | 1.12 |
| 998 | 1420 | 0.457 | 1036 | 0.334 | 0.27724 | 0.73 | 1.37 | 1944 | 0.099 | 0.11921 | 1.37 | 1.37 |
| 1001 | 851 | 0.233 | | | | | | 1803 | 0.317 | 0.00791 | 2.12 | 2.12 |
| 1065 | 1479 | 0.022 | | | | | | 1051 | 0.066 | 0.00244 | 0.71 | 1.41 |
| 1081 | 406 | 0.063 | 378 | 0.295 | 0.76098 | 0.93 | 1.07 | 416 | 0.290 | 0.91556 | 1.02 | 1.02 |
| 1172 | 13003 | 0.270 | 18022 | 0.434 | 0.22518 | 1.39 | 1.39 | 12548 | 0.233 | 0.82302 | 0.97 | 1.04 |
| 1195 | 2409 | 0.117 | 1711 | 0.173 | 0.02462 | 0.71 | 1.41 | 2739 | 0.247 | 0.58493 | 1.14 | 1.14 |
| 1215 | 1615 | 0.090 | 2335 | 0.218 | 0.05794 | 1.45 | 1.45 | 3749 | 0.175 | 0.00219 | 2.32 | 2.32 |
| | Control | | Lovastatin | | | | | Cholestyramine + Lovastatin | | | | |
| MSN | AVOL | CV | AVOL | CV | PROB | RATIO | NFOLD | AVOL | CV | PROB | RATIO | NFOLD |
| 34 | 29657 | 0.079 | 28727 | 0.041 | 0.54269 | 0.97 | 1.03 | 22570 | 0.121 | 0.00267 | 0.76 | 1.31 |
| 97 | 13102 | 0.033 | 15633 | 0.033 | 0.00013 | 1.19 | 1.19 | 18773 | 0.113 | 0.00064 | 1.43 | 1.43 |
| 99 | 25928 | 0.088 | 25223 | 0.101 | 0.65931 | 0.97 | 1.03 | 23633 | 0.204 | 0.63399 | 0.91 | 1.10 |
| 104 | 28850 | 0.080 | 28377 | 0.104 | 0.77967 | 0.98 | 1.02 | 31576 | 0.124 | 0.21585 | 1.09 | 1.09 |
| 115 | 17488 | 0.118 | 13656 | 0.216 | 0.04276 | 0.78 | 1.28 | 16209 | 0.117 | 0.33788 | 0.93 | 1.08 |
| 122 | 22469 | 0.138 | 21474 | 0.125 | 0.60653 | 0.96 | 1.05 | 24432 | 0.066 | 0.24256 | 1.09 | 1.09 |
| 142 | 27870 | 0.046 | 24557 | 0.063 | 0.00638 | 0.88 | 1.13 | 23328 | 0.092 | 0.00391 | 0.84 | 1.19 |
| 147 | 27000 | 0.058 | 22469 | 0.073 | 0.00247 | 0.83 | 1.20 | 24040 | 0.079 | 0.02647 | 0.89 | 1.12 |
| 178 | 9747 | 0.044 | 8696 | 0.029 | 0.00194 | 0.89 | 1.12 | 7780 | 0.094 | 0.00115 | 0.80 | 1.25 |
| 182 | 7642 | 0.087 | 6877 | 0.139 | 0.17797 | 0.90 | 1.11 | 5480 | 0.145 | 0.00194 | 0.72 | 1.39 |
| 191 | 10964 | 0.074 | 15227 | 0.133 | 0.00273 | 1.39 | 1.39 | 12862 | 0.082 | 0.01277 | 1.17 | 1.17 |
| 204 | 6268 | 0.144 | 3198 | 0.161 | 0.00036 | 0.51 | 1.96 | 3401 | 0.472 | 0.00830 | 0.54 | 1.84 |
| 232 | 8495 | 0.214 | 5047 | 0.101 | 0.00380 | 0.59 | 1.68 | 6059 | 0.153 | 0.02731 | 0.71 | 1.40 |
| 275 | 4715 | 0.080 | 2818 | 0.290 | 0.02274 | 0.60 | 1.67 | 5518 | 1.034 | 0.81501 | 1.17 | 1.17 |
| 279 | 4852 | 0.040 | 3751 | 0.160 | 0.00478 | 0.77 | 1.29 | 3837 | 0.307 | 0.09150 | 0.79 | 1.26 |
| 322 | 13385 | 0.150 | 15448 | 0.129 | 0.16677 | 1.15 | 1.15 | 16408 | 0.134 | 0.06933 | 1.23 | 1.23 |
| 361 | 5331 | 0.167 | 7855 | 0.189 | 0.02018 | 1.47 | 1.47 | 12011 | 0.315 | 0.01117 | 2.25 | 2.25 |
| 365 | 3264 | 0.045 | 3396 | 0.165 | 0.62819 | 1.04 | 1.04 | 3351 | 0.165 | 0.74027 | 1.03 | 1.03 |
| 367 | 11735 | 0.114 | 27001 | 0.115 | 0.00006 | 2.30 | 2.30 | 19156 | 0.094 | 0.00021 | 1.63 | 1.63 |
| 395 | 5406 | 0.116 | 4836 | 0.186 | 0.27845 | 0.89 | 1.12 | 3497 | 0.170 | 0.00148 | 0.65 | 1.55 |
| 413 | 2983 | 0.157 | 9771 | 0.192 | 0.00017 | 3.28 | 3.28 | 19195 | 0.370 | 0.00126 | 6.44 | 6.44 |
| 423 | 4320 | 0.100 | 2967 | 0.187 | 0.00296 | 0.69 | 1.46 | 3531 | 0.404 | 0.27010 | 0.82 | 1.22 |
| 461 | 3606 | 0.072 | 3281 | 0.161 | 0.31176 | 0.91 | 1.10 | 3017 | 0.406 | 0.61548 | 0.84 | 1.20 |
| 475 | 4565 | 0.135 | 5391 | 0.057 | 0.02680 | 1.18 | 1.18 | 4251 | 0.314 | 0.64874 | 0.93 | 1.07 |
| 479 | 2492 | 0.199 | 2351 | 0.108 | 0.62542 | 0.94 | 1.06 | 5690 | 0.123 | 0.00057 | 2.28 | 2.28 |
| 490 | 5649 | 0.052 | 5122 | 0.257 | 0.53802 | 0.91 | 1.10 | 5302 | 0.483 | 0.82210 | 0.94 | 1.07 |
| 502 | 3386 | 0.068 | 3304 | 0.165 | 0.76152 | 0.98 | 1.02 | 2898 | 0.143 | 0.04862 | 0.86 | 1.17 |
| 556 | 3004 | 0.167 | 2632 | 0.154 | 0.23234 | 0.88 | 1.14 | 2896 | 0.147 | 0.72336 | 0.96 | 1.04 |
| 578 | 2677 | 0.141 | 3213 | 0.123 | 0.05729 | 1.20 | 1.20 | 3650 | 0.078 | 0.00210 | 1.36 | 1.36 |
| 590 | 3745 | 0.262 | 3184 | 0.219 | 0.32886 | 0.85 | 1.18 | 3136 | 0.262 | 0.31959 | 0.84 | 1.19 |
| 602 | 3147 | 0.090 | 3415 | 0.086 | 0.17703 | 1.09 | 1.09 | 3099 | 0.042 | 0.74015 | 0.98 | 1.02 |
| 610 | 3055 | 0.250 | 4575 | 0.089 | 0.00465 | 1.50 | 1.50 | 5607 | 0.309 | 0.01630 | 1.84 | 1.84 |
| 625 | 3493 | 0.233 | 3152 | 0.110 | 0.58195 | 0.90 | 1.11 | 3106 | 0.415 | 0.59038 | 0.89 | 1.12 |
| 633 | 2750 | 0.102 | 4489 | 0.054 | 0.00005 | 1.63 | 1.63 | 3110 | 0.309 | 0.54935 | 1.13 | 1.13 |
| 646 | 2957 | 0.086 | 2896 | 0.179 | 0.82942 | 0.98 | 1.02 | 2501 | 0.058 | 0.02013 | 0.85 | 1.18 |
| 664 | 1115 | 0.091 | 1198 | 0.050 | 0.15411 | 1.07 | 1.07 | 1224 | 0.122 | 0.21464 | 1.10 | 1.10 |
| 984 | 594 | 0.078 | 632 | 0.274 | 0.69042 | 1.06 | 1.06 | 661 | 0.073 | 0.09189 | 1.11 | 1.11 |
| 998 | 1420 | 0.457 | 2614 | 0.084 | 0.00483 | 1.84 | 1.84 | 2218 | 0.259 | 0.07136 | 1.56 | 1.56 |
| 1001 | 851 | 0.233 | 2060 | 0.234 | 0.00116 | 2.42 | 2.42 | 5507 | 0.413 | 0.00219 | 6.47 | 6.47 |
| 1065 | 1479 | 0.022 | 885 | 0.098 | 0.00242 | 0.60 | 1.67 | 1128 | 0.380 | 0.35269 | 0.76 | 1.31 |
| 1081 | 406 | 0.063 | | | | | | 756 | 0.009 | 0.00197 | 1.86 | 1.86 |
| 1172 | 13003 | 0.270 | 16344 | 0.314 | 0.26325 | 1.26 | 1.26 | 20874 | 0.095 | 0.00272 | 1.61 | 1.61 |
| 1195 | 2409 | 0.117 | 3391 | 0.158 | 0.01733 | 1.41 | 1.41 | 4801 | 0.104 | 0.00043 | 1.99 | 1.99 |
| 1215 | 1615 | 0.090 | 2454 | 0.299 | 0.10481 | 1.52 | 1.52 | 3017 | 0.323 | 0.05940 | 1.87 | 1.87 |

Since all p value cut-offs represent a somewhat arbitrary threshold, it is possible and likely to miss significant protein markers using one embodiment of the present invention. However, by looking at related agents, which may be related by chemical structure or mechanism of action, one can find proteins with altered abundance with respect to the controls. Even though not statistically significant alone, if such a protein were found to be altered in biological samples from animals treated with slightly different but similarly acting agents, the result can be statistically significant. When determining what is to be considered a protein marker, a protein may constitute a marker of efficacy or toxicity for an agent even when not statistically significant in a single experiment with one agent alone.

Identification of a protein marker may be performed by detecting proteins with altered abundance for multiple similar agents. The similarities may be chemical structure, function or physiological or toxic effect. Testing with agents having common mechanisms of action is particularly preferred for markers comparing related agents. An ideal example is screening new compounds and comparing their marker changes to those of a standard pharmaceutical having the same general usage.

For example, methionine adenosyltransferase has a p value above 0.001 for all of the agents tested. If one required such a stringent confidence level, this marker would be ignored. However for fluvastatin, it is 0.00234, for probucol, it is 0.00139, for pravastatin sodium, it is 0.00425 and for lovastatin, it is 0.00307. Thus, this protein is an acceptable protein marker due to its altered level in biological samples from animals treated with multiple related drugs without a need to raise the p value. This situation is not unique and may be found in many other markers. Representative examples are listed in Table 5.

TABLE 5

| | | | | |
|---|---|---|---|---|
| Annexin VI | pravastatin | 0.00202 | lovastatin | 0.00143 |
| MSN 76 | fluvastatin | 0.00161 | lovastatin | 0.00150 |
| MSN 143 | fluvastatin | 0.00188 | lovastatin | 0.00353 |
| MSN 154 | probucol | 0.00365 | simvastatin | 0.00408 |
| MSN 172 | simvastatin | 0.00468 | fluvastatin | 0.00299 |
| MSN 229 | simvastatin | 0.00182 | pravastatin | 0.00176 |
| MSN 371 | fluvastatin | 0.00394 | simvastatin | 0.00188 |

Other examples include MSN 117, 339, 497, 506, 665, 777, 934 and others.

When determining what is to be considered a protein marker, combinations of proteins may constitute a combination marker of efficacy or toxicity for an agent. Even when two or more proteins are not sufficiently statistically significant to be considered markers by themselves, when considered in combination, the combination marker may be statistically significant. This is done by determining proteins which are at altered abundances in biological samples from animals treated with an agent of interest and control biological samples from animals not treated with an agent of interest. Selecting two proteins that are less than statistically significant markers by themselves, one may combine the values for two or more of these proteins and determine whether the combination of values is altered in a statistically significant manner. Combination markers result when statistically significant differences between biological samples from treated animals and biological samples from untreated animals are determined. Suitable data-mining reveals a number of combination markers, and the theoretical rationale for some of these combination markers is still being determined.

Testing with agents having different mechanisms of action is particularly preferred when searching for new agents of potentially new mechanisms of action. This is searching by purely secondary pharmaceutical function. By comparing protein markers across different agents, less than statistically significantly changed proteins may become protein markers. Both processes were used with the antilipemic agents in the present invention.

Through suitable data mining techniques, one may even determine combination markers across the spectrum of different agents such that even combination markers, which are not statistically significant then, become significant considering their determination in multiple related agents.

An index marker is similar to a combination marker except that each protein in the index is itself already statistically significant as a protein marker alone. An index marker is an aggregate of plural significant protein markers which taken together and compared to the same index marker of a different sample. The index marker is then an extremely significant combination. For example, using a combination of markers, each with p<0.001, may yield an index marker of p<0.00001 or lower.

Protein markers found across drugs in different categories of modes of actions producing the same markers are perhaps the best markers for screening new drugs for a given indication because they are not mechanism of action specific. These are believed to reveal elements common to the mechanisms of action of the different pharmacological classes. Such a marker is good for screening for drugs having completely unknown modes of action but directed to a similar disease treatment objcetive.

By using a different method for measuring the proteins on a two-dimensional electrophoretic gel, different markers may also be uncovered. Furthermore, by comparing how one protein changes in abundance with respect to others, still other protein markers may be found. For example, protein MSN 261 was also changed together with (i.e., its abundance in a drug treatment experiment is correlated with) HMGCoA synthase (cytosolic), HMGCoA synthase (mitochrodrial), HMGCoA synthase (cytosolic) (other form) and IPP-isomerase. Although MSN 261 has a P value of >0.005 for all drugs tested, it is still considered a marker because of this strong correlation with other markers found by In view of this data, one may conclude that protein MSN 261 is at least a protein marker, and likely to be a protein in the biosynthetic pathway for cholesterol.

This method is performed by comparing all proteins that change in abundance in the same or opposite direction as known protein markers. Even if the change in abundance of the proposed protein marker is not changed significantly, the fact that its abundance changes along with established protein markers indicates it may be an acceptable marker.

Another method for finding a marker even when the data is not statistically significant is to determine whether a protein is altered in tandem with known protein markers. Proteins which are not sufficiently altered to be considered protein markers are called protein “submarkers” when they have altered levels in tandem direction and magnitude when consistent among a group of samples. Essentially the same experimental methodology is performed as above for finding a protein marker for efficacy or toxicity for an agent. The direction and amount of alteration between the control and agent treated samples is noted. This is compared across multiple individuals and compared to established protein markers. Tandem moving protein submarkers which are altered both in direction and in amount between individuals and paralleling known protein markers may then be considered to be “protein markers” in their own right. Such may then be assayed for the multitude of purposes as any other marker.

Another method for measuring the proteins in a two-dimensional electrophoretic gel is by determining qualitatively whether a protein is present or absent. For example, a protein found in a biological sample from a control but not in a comparable sample from an agent exposed tissue would be of particular interest as it represents that the agent eliminated the protein completely. Likewise, the reverse where a protein is induced only in treated but not controls is also of particular interest. A p value is not even calculable in these situations as one is comparing to zero.

Protein spot MSN 204, alanine aminotransferase, is present in controls but is eliminated in samples treated with antilipemic agents. A decrease may be seen in low dose treatments for some individuals.

Protein spot MSN 1255 has the reverse behavior by usually being absent in controls and sometimes even in samples from low dose of antilipemic agents treatments. However, in high dosages, this protein is consistently present.

Another qualitative or quantitative change in protein marker levels is in the presence of or amount of protein variants. Some drugs are known to alter glycosylation and the agent being tested may induce a different abundance of protein variants. Likewise, cleavage fragments (or the lack thereof) may be in altered abundance. Still further, enzymes may be in the same concentration but have dramatically different activity due to the agent. In all of these situations, the altered level or change in abundance of a protein or its variant(s) may be used to serve as a suitable marker for efficacy or toxicity. This may be observed as a shift in spot location or new spot formation.

Not only can the present invention determine response to an agent after treatment has begun, but also susceptibility to toxicity with an agent or effective response to treatment with the agent may be determined. Furthermore, some indication as to the appropriate dosage may be given. This is done, by measuring toxicity or efficacy susceptibility markers in a biological sample from a test tissue of interest before treatment begins.

The proteins in the biological sample from an agent treated organism or tissue may be tested against a number of other groups depending on the data desired. The simplest comparison is to untreated controls. However, comparisons to positive and negative treated controls may also be performed. In that situation, the positive controls include samples from treatments with an agent having the same mechanism of action and agents having a different mechanism but the same general effect. Negative treated controls may be from samples treated with an agent with the same mechanism of action but having an opposite effect or samples treated with agents having an unrelated mechanism. To best determine which agents have an unrelated mechanism, it is desirable to compare to a composite effect of many drugs and other agents, preferably from a pharmaceutical proteomics large database. The comparison to the positive control same mechanism of action and the negative control same mechanism of action may be seen as agonist/antagonist effects and correlations between these two control groups provides a further source for protein markers.

Furthermore, the toxicity controls may be further subdivided into toxicity controls having been treated with an agent having the same mechanism of action, with an opposite mechanism of action and by an unrelated mechanism of action. As before, the unrelated mechanism of action control is best determined from a large database of many different and unrelated agents such as a large pharmaceutical proteomics database. Also as before, the controls with opposite mechanisms of action may be correlated to each other for providing a further source of protein markers. Still furthermore, plural (or all possible) comparisons between the test sample and plural controls are preferable.

Total protein markers identified are listed below. New protein markers listed below are those that are provably unknown proteins or ones for which evidence to date does not suggest that they are known. Some of the markers gave insufficient or conflicting information and are considered unknown for the purposes of the present invention. As will be shown in the examples, protein identity was determined by molecular weight, pI, molecular mass of digested peptides and fragment ions, partial or complete sequence of the peptides or entire protein, etc. In some situations, the molecular mass determined by MALDI conflicted with the determination achieved by electrospray MS. This may be due to a number of factors including poorly resolved spots on the gel, experimental conditions, etc. Conflicting data is not considered an identification and thus considered to be “unknown”. Examples of MALDI and electrospray data is given in Table 6. “Unknown” is defined as not being listed in the public NCBI non-redundant gene sequence database or the SwissProt database. Examples of MALDI and electrospray MS data for selected proteins is given in Table 7.

TABLE 6
Peptide Molecular Mass For MALDI

| MSN | 372 | 297 | 806 | 34 |
|---|---|---|---|---|
| Sample | 47 | 56 | 18 | 4 |
| Peaks | 915.0180 | 855.9430 | 1013.0280 | 1488 |
| Single | 1105.0729 | 956.9130 | 1181 | 1491.2190 |
| Charged | 1123.0769 | 1197.0979 | 1390.0409 | 1505.6179 |
| Ions | 1559.1079 | 1327.0189 | 1634.1120 | 1521.5910 |
| | 1778.1580 | 1664.9900 | 2029.0600 | 1629.7110 |
| | 1949.1869 | 1723.1559 | | 2316.4288 |
| | 1965.0979 | 1844.0900 | | 2380.1 |
| | 1981.0690 | 1972.1060 | | 2193.1 |
| | 2398.2269 | 2547.0929 | | 2366.1 |
| | 2576.1268 | 1951.1 | | |
| | 2681.0890 | | | |
| | 2593.2 | | | |
| | 2695.1 | | | |
| | 2723.2 | | | |
| | 872.1 | | | |

Peptide Molecular Mass For Electrospray

| MSN | 372 | 297 | 34 |
|---|---|---|---|
| Multiply | 375.1 | 442.9 | 496.7 |
| Charged | 458 | 575.6 | 753.1 |
| Ion Mass | 553 | 598.9 | 761.3 |
| M/Z | 562 | 663.9 | 805.2 |
| For | 593.6 | 833.2 | |
| MS/MS | 650.6 | 850 | |
| | 780 | 855.4 | |
| | 800.5 | 862.1 | |
| | | 922.6 | |
| | | 986.6 | |
| | | 1274.8 | |

TABLE 7
MALDI And Electrospray Data for Selected Spots

| | | | | | MALDI^{e.)} | | ESI^{f.)} | |
|---|---|---|---|---|---|---|---|---|
| MSN^{a)} | Protein Name | Accession #^{b.)} | MW^{c).} | pl^{d.)} | # M. Pep.^{g.)} | % S. C.^{h.)} | # M. Pep. | % S.C. |
| 138 | 4-Hydroxyphenylpyruvate | gi 3435296 | 45112 | 6.29 | 12 | 33 | 3 | 11 |
| | dioxygenase | | | | | | | |
| 252 | Serine protease inhibitor 2 | sp P05545 | 43773 | 5.39 | 12 | 40 | 7 | 24 |
| 305 | Phenylalanine hydroxylase | sp P04176 | 51821 | 5.76 | 15 | 42 | 12 | 26 |
| | (EC 1.14.16.1) | | | | | | | |
| 361 | HMG-CoA synthase, | sp P22791 | 52714 | 8.24 | | | 4 | 8 |
| | mitochondrial frag. (EC 4.1.3.5) | | | | | | | |
| 463 | Apolipoprotein A-I | sp P04639 | 27394 | 5.51 | 14 | 43 | 6 | 20 |
| 490 | Alpha 2u-globulin | sp P02761 | 18730 | 5.48 | | | 6 | 40 |
| 532 | Protein kinase C inhibitor | sp P35214 | 28171 | 4.8 | 4 | 21 | 2 | 4 |
| 577 | Fructose-1,6-bisphosphatase | sp P19112 | 39478 | 5.54 | | | 5 | 10 |
| | (EC 3.1.3.11) | | | | | | | |
| 590 | Glucose-6-phosphate | sp P05370 | 59244 | 5.97 | | | 7 | 16 |
| | 1-dehydrogenase (EC 1.1.1.49) | | | | | | | |
| 610 | 3-Mercaptopyruvate | sp P97532 | 32809 | 5.88 | | | 5 | 17 |
| | sulfotransferase | | | | | | | |
| 664 | Major vault protein | sp Q62667 | 98504 | 5.65 | | | 11 | 18 |
| 932 | Annexin IV | sp P55260 | 35743 | 5.32 | 7 | 41 | 6 | 23 |
| 993 | Induced in androgen-indep. | gi 456282 | 35866 | 5.6 | | | 3 | 12 |
| | prostate cells by eff. of apopt. | | | | | | | |
| 1119 | Isopentenyl-diphosphate | sp O35760 | 26402 | 5.57 | 6 | 14 | 6 | 20 |
| | delta-isomerase (EC 5.3.3.2) | | | | | | | |
| 34 | Unknown | | | | 6 good pept. | | 4 good spec. | |
| 142 | Ketohexokinase (ID before) | sp Q02974 | 32750 | 6.24 | 6 | 24 | 2 | 7 |
| 168 | Antiquitin - rat fragment | gi 1083595 | 25158 | 7.73 | 5 | 16 | 2 (4 hits) | 14 |
| 178 | Aminoacylase | Sp q03154 human | 45885 | 5.77 | 5 | 13 | 3 | 10 |
| 182 | Fructose-1,6-biphosphatase | sp p19112 | 39584 | 5.54 | 11 | 29 | 1 (2 hits) | |
| 297 | Unknown | | | | 9 good pept. | | 11 good spe | |
| 321 | Glutathione synthetase | sp p46413 | 52345 | 5.49 | 7 | 21 | 4 | 10 |
| 372 | Unknown | | | | 11 good pept. | | 8 good spe. | |
| 457 | Annexin VI | sp p48037 | 75755 | 5.39 | 13 | 20 | 1 | |
| 698 | Cytokeratin ends A | Sp Q10758 | 53887.56 | 5.82 | | | 7 | 15 |
| 806 | Unknown | | | | 5 good pept. | | 2 good spe. | |
| 933 | HMG-CoA synthase (ID before) | sp P17425 | 57434 | 5.59 | 7 | 14 | | |
| 934 | Ras-GTPase-activating protein | gi 1902907 | 51829 | 5.41 | | | 4 | 14 |
| | SH3-domain binding | | | | | | | |
| | protein (mouse) | | | | | | | |

Even though the protein may not be heretofore isolated or characterized, the present invention effectively isolates and characterizes the proteins. From the MSN number given below, one has a unique isolated protein from a spot on the 2-dimensional electrophoretic gel. The relative molecular weight and relative pI for each spot are determinable by reference to established landmark proteins which are fully characterized by sequencing and a theoretical molecular weight and pI calculated. By plotting the theoretical values on a graph and comparing the location of the previously unknown spot, these identifying features are determined. See Anderson et al, Electrophoresis 16:1977-1981 (1995) for more details, the contents of which are specifically incorporated by reference. This provides a reproducible method for isolating the protein markers of the present invention.

The protein markers which are perturbed by antilipemic agents are as follows. When different variants of the proteins are present and used as markers, references to the different MSN numbers is given.

TABLE 8
Total Protein Markers

Actin gamma
Adenosine kinase (EC 2.7.1.20)
Adensylhomocysteinase
Alanine aminotransferase
Alpha 2u-globulin
Annexin IV
Annexin VI
Antiquitin
Apolipoprotein A-I
Apolipoprotein E precursor
Catechol O-methyl transferase
Calreticulin
Catalase
Cytokeratin ends A
N-G, N-G-dimethylarginine dimethylaminohydrolase
D-dopachrome tautomerase
Epoxide hydrolase, soluble
ER60 protease; 58 kD microsomal protein
Fatty acid binding protein, liver
Fructose-1,6-bisphosphatase (EC 3.1.3.11) (MSN 79)
Fructose-1,6-bisphosphatase (EC 3.1.3.11) (MSN 182)
Fructose-1,6-bisphosphatase (EC 3.1.3.11) (MSN 577)
Fumarylacetoacetate hydrolase
75 kD glucose related protein
Glucose-6-phosphate 1-dehydrogenase (EC 1.1.1.49)
Glutathione synthetase
90 kD heat shock protein
Heme oxygenase-1
Heterogeneous nuclear ribonucleoprotein K
HMG-CoA synthase, mitochondrial frag. (EC 4.1.3.5)
HMG-CoA synthase, cytosolic (EC 4.1.3.5) (MSN 413)
HMG-CoA synthase, cytosolic (EC 4.1.3.5) (MSN 933)
HMG-CoA synthase, (MSN 1250)
HumorF06
N-hydroxyarylamine sulfotransferase (EC 2.8.2.—)
3-Hydroxyanthranilate 3,4-dioxygenase (EC 1.13.11.6)
4-Hydroxyphenylpyruvate dioxygenase
Induced in androgen-indep. prostate cells by eff. of apopt.
Isopentenyl-diphosphate delta-isomerase (EC 5.3.3.2)
Isovaleryl-CoA dehydrogenase
Keratin type II cytoskeletal 8 (MSN 97)
Keratin type I cytoskeletal 18
Keratin type II cytoskeletal 8 (MSN 41)
Ketohexokinase (EC 2.7.1.3)
Lamin b
Major vault protein
Methionine adensyltransferase
3-Mercaptopyruvate sulfotransferase (EC 2.8.1.2)
23 kD Morphine-binding protein
Nucleolar phosphoprotein B23 (MSN 574)
Nucleolar phosphoprotein B23 (MSN 671)
2-oxoisovalerate dehydrogenase alpha subunit, mitochrondrial
Peroxisomal enoyl hydratase-like protein
Phenylalanine hydroxylase (EC 1.14.16.1)
Protein kinase C inhibitor
Pyruvate kinase, isoenzymes (MSN 282)
Pyruvate kinase L
Ras-GTPase-activating protein SH3-domain binding protein
Senescence marker protein-30 (MSN 55)
Senescence marker protein-30 (MSN 103)
Serine protease inhibitor 2
Tropomysin
MSN 34, MSN 42, MSN 59, MSN 66, MSN 69, MSN 73, MSN 76,
MSN 83, MSN 117, MSN 122, MSN 127, MSN 128, MSN 139,
MSN 143, MSN 148, MSN 154, MSN 155, MSN 197, MSN 203,
MSN 204, MSN 218, MSN 229, MSN 232, MSN 237, MSN 238,
MSN 261, MSN 267, MSN 268, MSN 275, MSN 279, MSN 286,
MSN 270, MSN 289, MSN 292, MSN 297, MSN 310, MSN 311,
MSN 318, MSN 322, MSN 339, MSN 347, MSN 350, MSN 358,
MSN 362, MSN 365, MSN 371, MSN 372, MSN 379, MSN 384,
MSN 395, MSN 399, MSN 416, MSN 420, MSN 423, MSN 427,
MSN 434, MSN 435, MSN 438, MSN 461, MSN 469, MSN 479,
MSN 492, MSN 497, MSN 502, MSN 506, MSN 510, MSN 522,
MSN 546, MSN 556, MSN 557, MSN 565, MSN 569, MSN 571,
MSN 578, MSN 602, MSN 605, MSN 613, MSN 618, MSN 625,
MSN 633, MSN 637, MSN 644, MSN 646, MSN 653, MSN 665,
MSN 666, MSN 669, MSN 681, MSN 689, MSN 718, MSN 719,
MSN 721, MSN 777, MSN 779, MSN 787, MSN 802, MSN 806,
MSN 810, MSN 839, MSN 876, MSN 879, MSN 887, MSN 888,
MSN 900, MSN 905, MSN 966, MSN 984, MSN 1001, MSN 1065,
MSN 1081, MSN 1053, MSN 1172, MSN 1195, MSN 1215,
and MSN 1255.

TABLE 9
New Protein Markers

MSN 34, MSN 42, MSN 59, MSN 66, MSN 69, MSN 73, MSN 76,
MSN 83, MSN 117, MSN 122, MSN 127, MSN 128, MSN 139,
MSN 143, MSN 148, MSN 154, MSN 155, MSN 197, MSN 203,
MSN 204, MSN 218, MSN 229, MSN 232, MSN 237, MSN 238,
MSN 261, MSN 267, MSN 268, MSN 275, MSN 279, MSN 286,
MSN 270, MSN 289, MSN 292, MSN 297, MSN 310, MSN 311,
MSN 318, MSN 322, MSN 339, MSN 347, MSN 350, MSN 358,
MSN 362, MSN 365, MSN 371, MSN 372, MSN 379, MSN 384,
MSN 395, MSN 399, MSN 416, MSN 420, MSN 423, MSN 427,
MSN 434, MSN 435, MSN 438, MSN 461, MSN 469, MSN 479,
MSN 492, MSN 497, MSN 502, MSN 506, MSN 510, MSN 522,
MSN 546, MSN 556, MSN 557, MSN 565, MSN 569, MSN 571,
MSN 578, MSN 602, MSN 605, MSN 613, MSN 618, MSN 625,
MSN 633, MSN 637, MSN 644, MSN 646, MSN 653, MSN 665,
MSN 666, MSN 669, MSN 681, MSN 689, MSN 718, MSN 719,
MSN 721, MSN 777, MSN 779, MSN 787, MSN 802, MSN 806,
MSN 810, MSN 839, MSN 876, MSN 879, MSN 887, MSN 888,
MSN 900, MSN 905, MSN 966, MSN 984, MSN 1001, MSN 1065,
MSN 1081, MSN 1053, MSN 1172, MSN 1195, MSN 1215,
and MSN 1255.

When combinations of lipid lowering drugs are used, some have little effect, some an additive effect and some a synergistic effect. In the present examples, the combination of cholestyramine and lovastatin gave the largest effect on major protein markers that are indicative of or result from the treatment. While considerable interanimal variability was observed leading to a high CV and thus higher P value, the absolute change was greatest. A quick method for reading this is to compare the ratios to the controls. This is believed to be due to different modes of action of these two drugs. Combinations of pharmaceutical compounds in a composition may be prepared using known effective dosages of these known pharmaceuticals in their conventional dosages.

Susceptibility markers include the detection of genetic polymorphism(s) resulting in an amino acid sequence variant in all or some of the protein. The agent will interact differently depending on the polymorphism(s) present. In addition, polymorphism(s) inside or outside the coding region of a gene may result in different levels of expression. The protein markers may be involved in the metabolic pathway or they may be non-specific drug metabolism or repair mechanisms. For example having a protein variant in a component of the cytochrome P-450 isoenzyme system is well known to alter an individual's response to certain drugs by altering the metabolism rate (% of compound used by enzyme and/or turnover rate) and thus bioavailability. Superoxide dismutase and catalase variants appear to affect the ability of one to repair damage from free oxygen radicals and hydrogen peroxide respectively, generated by or directly from certain agents.

Absolute determination of an acceptable response by measuring susceptibility marker(s) may be due to non-genetic factors as well. Normal physiological changes due to time of day, recent foods consumed, exposure to other environmental agents or other drugs etc. also cause physiological changes which alter marker proteins abundance.

Susceptibility markers are determined by comparing the proteins in a proteome from individuals known to respond well to the drug and individuals known to experience toxicity from the drug. This may be done in the same manner as other marker determination and likewise used in the same manner. Proteins that are increased or decreased above a statistically significant amount are deduced to be toxicity or efficacy susceptibility markers. While many of the differences may be too small to have any significant effect, adequate comparison reveals certain markers of susceptibility. Measuring such markers permits one to predetermine whether an agent is likely to be acceptable for the individual, species, breed or variety before treatment begins.

The diagnostic kits of the present invention are typically used in an “sandwich” format to detect the presence or quantity of proteins in a biological sample. A description of various immunoassay techniques is found in BASIC AND CLINICAL IMMUNOLOGY (4th ed. 1982 and more recent editions) by D. P. Sites at al., published by Lange Medical Publications of Los Altos, Calif., and in a large number of U.S. Patents including U.S. Pat. Nos. 3,654,090, 3,850,752 and 4,016,043, the respective contents of which are incorporated herein by reference.

In a preferred embodiment, the kit further includes, in a separate package, an amplifying reagent such as complement, like guinea pig complement, anti-immunoglobulin antibodies or S. aureus cowan strain protein A that reacts with the antigen or antibodies being detected. In these embodiments, the label specific binding agent is capable of specifically binding the amplifying means when the amplifying means is bound to the protein or antibody.

Important to the labeling and detection systems is the ability to determine quantity of label present to quantify the ligands present in the original sample. Since the signal and its intensity is a measure of the number of molecules bound from the sample and hence of the number of receptors bound, the number of ligand molecules in the original sample may be determined. Optical and electrical signals are readily quantifiable. Radioactive signals may also be quantifiable directly but preferably is determined optically by use of a standard scintillation cocktail.

While the receptors most commonly utilized are antibody molecules, or a portion thereof, one may equally use other specific binding receptors such as hormone receptors, certain cell surface proteins (also called RECEPTORS in the scientific literature), an assortment of enzymes, signal transduction and binding proteins found in biological systems.

Likewise ligands exemplified as proteins below may also be small organic molecules such as metabolic products in a cell. By simultaneously detecting many or all metabolites in a sample, one can determine the global effects of an effector on the cell. Effectors may be drugs, toxins, infectious agents, physiological stress, environmental changes, etc.

As the number of markers found is large, a simultaneous multiple assaying system such as a microarray of binding agents for each desired protein marker is preferred. In such a microarray, a specific binding receptor for each protein marker ligand, e.g. an antibody, is immobilized at a different address and contained in a distinct region of the microarray or bound to a distinct particle or label. The protein marker ligand containing sample is then contacted to the microarray and allowed to bind. Binding may then be detected by a number of techniques, known per se, particularly preferred being binding a labeled receptor to one or more components of a ligand/receptor complex and detecting the label.

Microarrays containing multiple receptors are known per se. An earlier discovery of a test strip with multiple receptors has been commercially used for decades. A number of designs for multiple simultaneous binding assays are known per se in the analytic testing field.

The array may utilize antibody or other receptor display phage as a binding agent or an immobilizing agent for the protein marker ligand. Either the receptor alone or the whole display phage may be used. When used as an immobilizing agent, different cells of the microarray contain a different phage. When used as a labeled binding agent, the phage may be labeled (before or after binding to the ligand) by a number of techniques (such as direct fluorescent dyes, e.g. TOTO-1, labeled protein A or G, labeled anti-Ig, etc.) and utilized without prior identification of which display phage contains a particular antibody as an initial immobilized capture receptor performs the discrimination.

The techniques described in provisional patent application 60/166,266 filed Nov. 18, 1999 of N. Leigh Anderson may be employed to measure a very large number of proteins simultaneously, including any or all of those in a pathway relating to efficacy or toxicity. Such a technique may be applied to detecting any or all of the protein markers of the present invention.

For microarrays which are not a unitary solid phase, multiple different beads, each with a different label or having a different combination of labels may be used. For example a bead having different shades of a chromagen or different proportions of different chromagens or other detectable features. Each bead or set of beads with the same identifying label(s) is to have an immobilized ligand or receptor. Individual sets of beads may be identified in a mixture by spreading on a flat surface and scanning or by moving the beads past a detector. The combination of the labels and the bead label(s) provides identification of the ligand of interest in the sample. The numerical ratio of beads having labels to beads without labels or with different labels provide a quantitative measurement. Just as the sample may be deduced from which addresses contained labels in a traditional microarray, with plural unique beads, the address may be deduced by determining which bead contains their corresponding label(s).

Pharmaceutical compositions may be prepared for use in humans or animals via the oral, parenteral or rectal route, in the form of wafer capsules, tablets, gelatin capsules, drinkable solutions, injectable solutions, including delayed forms and sustained-release dressings for transdermal administration of the active principle, nasal sprays, or topical formulations (cream, emulsion, etc.), comprising a derivative of a general formula according to the invention and at least one pharmaceutically acceptable carrier. The pharmaceutical compositions according to the invention are advantageously dosed to deliver the active principle in a single unit dose.

For oral administration, the effective unit doses are between 0.1 μg and 500 mg. For intravenous administration, the effective unit doses are between 0.1 μg and 100 mg. According to the invention, the pharmaceuticals are preferably administered orally, for example, in the form of tablets, dragees, capsules, solutions, or intraperitoneally, intramuscularly, subcutaneously, intraarticularly or intravenously, for example, by means of injection or infusion. It is especially preferred that the application according to the invention occurs in such a manner that the active agent is released with delay, that is as a depot.

Unit doses can be administered, for example, 1 to 4 times daily. The exact dose depends on the method of administration and the condition to be treated. Naturally, it can be necessary to vary the dose routinely depending on the age and the weight of the patient and the severity of the condition to be treated.

### EXAMPLE 1

### Preparation of 2-Dimensional Electrophoresis Gels

Male F344 rats (Charles River, Raleigh, N.C.), 8 weeks of age and weighing 167-182 g were housed individually in rat gang cages in an environmentally controlled room and were fed with Rodent Chow (Research Diets Inc., New Brunswick, N.J.) and tap water ad libitum. Three groups of five rats each received control feed, rodent chow milled with 16 ppm (approximately 1.6 mg/kg/day) lovastatin and rodent chow milled with 1500 ppm (approximately 150 mg/kg/day) lovastatin respectively for 7 days. The animals were guillotined after CO₂ asphyxiation on the day following the last treatment. Liver samples (150 mg of the left apical lobe) were removed and flash frozen in liquid nitrogen and kept at −80° C. until analysis.

The samples were homogenized in eight volumes of 9M urea, 2% CHAPS, 0.5% dithiothreitol (DTT) and 2% carrier ampholytes pH 8-10.5. The homogenates were centrifuged at 420,000×g at 22° C. for 30 min. (TL100 ultracentrifuge, TLA 100.3 rotor, 100,000 rpm (Beckman Instruments, Palo Alto, Calif.)). The supernatant was removed, divided into four aliquots and stored at −80° C. until analysis.

Ultrapure reagents for polyacrylamide gel preparation were obtained from Bio-Rad (Richmond, Calif.). Ampholytes pH 4-8 were from BDH (Poole, UK), ampholytes pH 8-10.5 were from Pharmacia (Uppsala, Sweden) and CHAPS was obtained from Calbiochem (La Jolla, Calif.). Deionized water from a high purity water system (Neu-Ion, Inc., Baltimore, Md.) was used. System filters are changed monthly to ensure 18 MΩ purity. HPLC grade methanol and glacial acetic acid were furnished from Fisher Scientific (Fair Lawn, NJ). HPLC grade acetonitrile was obtained from Baker (Phillipsburg, N.J.). Dithiothreitol (DTT) was obtained from Gallard-Schlesinger Industries, Inc. (Carle Place, NY). Iodoacetamide, ammonium bicarbonate, trifluoroacetic acid and α-cyano-4-hydroxycinnamic acid were obtained from Sigma Chemical Co. (St. Louis). Modified porcine trypsin was purchased from Promega (Madison, Wis.). All chemicals (unless specified) were reagent grade and used without further purification.

Sample proteins were resolved with two-dimensional gel electrophoresis using the 20×25 cm ISO-DALT® 2-D system (Anderson, 1991). 8 μl of solubilized sample were applied to each gel, and the gels were run for 25,050 volt-hours using a progressively increasing voltage with a high-voltage programmable power supply. An Angelique™ computer-controlled gradient-casting system (Large Scale Biology Corporation, Rockville, Md.) was used to prepare the second-dimension SDS slab gels. The top 5% of each gel was 11% T acrylamide and the lower 95% of the gel varied linearly from 11% to 19% T. The IEF gels were loaded directly onto the slab gels using an equilibration buffer with a blue tracking dye and were held in place with a 1% agarose overlay. Second-dimensional slab gels were run overnight at 160 V in cooled DALT tanks (10° C.) with buffer circulation and were taken out when the tracking dye reached the bottom of the gel. Following SDS electrophoresis, the slab gels were fixed overnight in 1.5 liters/10 gels of 50% ethanol/3% phosphoric acid and then washed three times for 30 min in 1.5 liters/10 gels of cold DI water. They were transferred to 1.5 liters/10 gels of 34% methanol/17% ammonium sulfate/3% phosphoric acid for one hour, and after the addition of one gram powdered Coomassie Blue G-250 the gels were stained for three days to achieve equilibrium intensity.

Stained slab gels were scanned and digitized in red light at 133 micron resolution, using an Eikonix 1412 scanner and images were processed using the Kepler® software system as described (Anderson '94). Groupwise statistical comparisons were made to search for treatment-related protein abundance changes.

### EXAMPLE 2

### Identification of Protein Markers

Gel pieces containing the proteins of interest were manually excised from a Coomassie stained gel and placed in a 96-well polypropylene microtiter plate. Samples were in-gel digested with trypsin according to the procedure of Shevchenko et al, Analytical Chemistry 68:850-858 (1996), with slight modifications. Briefly, the excised samples were destained by two 60 min cycles of bath sonication in 0.2 M NH₄HCO₃ in 50% CH₃CN with the resulting solution aspirated after each cycle. A volume of 0.2 M NH₄HCO₃ in 50% CH₃CN to sufficiently cover the gel pieces was added. Reduction and alkylation was accomplished by adding 135 nmol DTT and incubating at 37° C. for 20 min. After cooling, 400 nmol of iodoacetamide was added and incubated at room temperature in the dark for 20 min. The supernatant was removed and the samples were washed for 15 min in 0.2 M NH₄HCO₃ in 50% CH₃CN. The gel pieces were dried at 37° C. for 15 min and partially rehydrated with 5 μl 0.2 M NH₄HCO₃. After dispensing 3 μl of trypsin (30 ng/μl), the samples were incubated at room temperature for 5 min. A sufficient volume of 0.2M NH₄HCO₃ was added to ensure complete submersion of the gel pieces in the digestion buffer. Samples were incubated overnight at 37° C. All samples were acidified with 1 μl glacial acetic acid. Tryptic peptides were extracted by initially transferring the digest supernatant to a clean 96-well polypropylene microtiter plate with two subsequent extraction and transfer cycles of 60 μl of 60% CH₃CN, 1% glacial acetic acid. The combined extraction supernatant was dried and reconstituted in 6 μl 1% glacial acetic acid for subsequent mass spectral analysis.

All samples were prepared using a-cyano-4-hydroxycinnamic acid as the MALDI matrix utilizing the dried droplet method, Karas et al, Analytical Chemistry 60:2299-2301. The matrix solution was saturated in 40% CH₃CN, 0.1% trifluoroacetic acid (TFA) in water. The peptide solution (1.0 μl) was applied first to the smooth, sample plate target, then 1.0 μl of matrix solution was stirred in with a pipette tip and the sample allowed to air evaporate.

MALDI experiments were performed on a PerSeptive Biosystems Voyager-DE STR time-of-flight mass spectrometer (2.0 m linear flight path) equipped with delayed ion extraction. A pulsed nitrogen laser (Model VSL-337ND, Laser Science, Inc.) at 337.1 nm (<4 ns FWHM pulse width) was used for all of the data acquisition. Data was acquired in the delayed ion extraction mode using a 20 kV bias potential, a 6 kV pulse and a 150 ns pulsed delay time. Dual microchannel plate (Model 3040MA, Galileo Electro-Optics Corp.) detection was utilized in the reflector mode with the ion signal recorded using a 2-GHz transient digitizer (Model TDS 540C, Tektronix, Inc.) at a rate of 1 GS/s. All mass spectra represent signal averaging of 128 laser pulses. The performance of the mass spectrometer produced sufficient mass resolution to produce the isotopic multiplet for each ion species below mass-to-charge (m/z) of 3000. The data was analyzed using GRAMS/386 software (Galactic Industries Corp.).

All MALDI mass spectra were internally calibrated using masses from two trypsin autolysis products (monoisotopic masses 841.50 and 2210.10). Mass spectral peaks were determined based on a signal-to-noise (S/N) of 3. Two software packages, Protein Prospector and Profound, were used to identify protein spots. The rat and mouse nonredundant (nr) database consisting of SwissProt, PIR, GeneBank and OWL was used in the searches. Parameters used in the searches included proteins less than 100 kDa, greater than 4 matching peptides and mass errors less than 45 ppm.

For electrospray MS/MS a home-built microelectrospray interface similar to an interface described by Gatlin et al, Analytical Biochemistry 263:93-101 (1998) was employed. Briefly, the interface utilizes a PEEK micro-tee (Upchurch Scientific, Oak Harbor, WA) into which one stem of the tee is inserted a 0.025″ gold wire to supply the electrical connection. Spray voltage was 1.8 kV. A microcapillary column was prepared by packing 10 μm SelectPore particles (Vydac, Hesperia, CA) to a depth of 12 cm into a 75×360 μm fused silica capillary PicoTip (New Objectives, Cambridge, Mass.). The PicoTip has a 15 μm i.d. needle tip with an incorporated borosilicate glass frit. A 70 μl/min flow from a MAGIC 2002 HPLC solvent delivery system (Michrom BioResources, Auburn, Calif.) was reduced using a splitting tee to achieve a column flow rate of 450 nl/min.

Samples were loaded on-column utilizing an Alcott model 718 autosampler (Alcott Chromatography, Norcross, Ga.). HPLC flow was split prior sample loop injection. Samples prepared for MALDI were further diluted 1:3 in 0.5% HOAc, and 2 μl of each sample was injected on-column. Using contact closures, the HPLC triggered the autosampler to make an injection and after a set delay time, triggered the mass spectrometer to start data collection.

A 12 min gradient of 5-55% solvent B (A: 2% ACN/0.5% HOAc, B: 90% ACN/0.5% HOAc) was selected for separation of trypsin digested peptides. Peptide analyses were performed on a Finnigan LCQ ion trap mass spectrometer (Finnigan M A T, San Jose, Calif.). The heated desolvation capillary was set at 150° C., and the electron multiplier at −900 V. Spectra were acquired in automated MS/MS mode with a relative collision energy (RCE) preset to 35%. To maximize data acquisition efficiency, the additional parameters of dynamic exclusion, isotopic exclusion and “top 3 ions” were incorporated into the auto-MS/MS procedure. For the “top 3 ions” parameter, an MS spectrum was taken followed by 3 MS/MS spectra corresponding to the 3 most abundant ions above threshold in the full scan. This cycle was repeated throughout the acquisition. The scan range for MS mode was set at m/z 375-1200. A parent ion default charge state of +2 was used to calculate the scan range for acquiring tandem MS.

Automated analysis of peptide tandem mass spectra was performed using the SEQUEST computer algorithm (Finnigan M A T, San Jose, Calif.). The non-redundant (NR) protein database was obtained as an ASCII text file in FASTA format from the National Center for Biotechnology Information (NCBI).

The 2DGE protein pattern of rat liver illustrates over 1000 Coomassie Blue stained protein spots. Lovastatin treatment altered the abundance of 66 liver proteins, based on the application of the two-tailed Student's t-test (1 new, one lost, 8 with p<0.0001 and 32 with p<0.001 and 64 with p<0.005). All the statistically significant changes occurred in the group receiving 1500 ppm lovastatin in feed for 7 days, an amount similar to the high dose used in the 24-month carcinogenicity study in rats (PDR). Changes were evident in livers of rats treated with 16 ppm lovastatin for 7 days, an exposure comparable to the maximum recommended daily dose in humans, but were not of statistical significance. The proteins affected by the treatment are indicated with spot numbers and protein name in Table 1. Several proteins have been identified in the F344 rat liver reference 2-D pattern published previously (Anderson et al, Electrophoresis 16:1977-1981 (1995). These spots were previously identified by a variety of techniques. Many of the spots that were not yet identified and were strongly affected by lovastatin treatment were subjected to tryptic-digestion and identified by MALDI-MS and/or LC-MS/MS. The results are given in Tables 5 and 6 above.

### EXAMPLE 3

### Identification of Other Antilipemic Protein Markers

The methods of Example 1 and 2 were repeated with high and low doses of fluvastatin, simvastatin, pravastatin, niacin, gemfibrozil and probucol. For these experiments, only pharmaceutical grade compounds were used with the trademark identifying the source. Previous experiments indicated that so-called generic equivalents are not always equivalent. In each experiment, the low dose was equivalent to the daily human therapeutic dose. The results are given in Tables 1 and 2. The data from Example 2 is given as a separate column for comparison. Across compound data is presented in these tables where the protein markers with a significance of p<0.001 and of p<0.005 are indicated.

### EXAMPLE 4

### Indentification of Further Protein Markers

The methods of Example 1, 2 and 3 were repeated with, lovastatin, cholestyramine, high cholesterol diet and a combination of lovastatin and cholestyramine. In each experiment, the dose was equivalent to slightly higher than the maximal human therapeutic dose. The rats were somewhat older and slight experimental protocol differences were used and thus the data is not directly comparable to that in Examples 1-3. Across agent data is presented in Tables 3 and 4.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

1-61. (canceled)

62. A method for determining whether a combination of proteins together form a protein marker of efficacy or toxicity for an agent when the proteins individually are not markers with a desired level of statistical significance, comprising;
determining proteins which are at altered levels in biological samples from an animal treated with an agent of interest and control biological samples from an animal not treated with an agent of interest, which proteins are less than the desired level for statistically significant markers by themselves,
selecting two or more of said proteins,
combining the values for two or more of said proteins and determining whether the combination of values is altered in a statistically significant manner,
wherein said combination of proteins results in the desired level of statistically significant differences between biological samples from treated animals and biological samples from untreated animals.

63. The method of claim 62 wherein said agent of interest and said related agent of interest are chemically related.

64. The method of claim 62 wherein said agent of interest and said related agent of interest have at least one common mechanism of action.

65. The method of claim 62 wherein said group of related agents are antilipemic agents.

66. The method of claim 62 wherein said related agent of interest is a drug used for comparable indications but functions by a different intended mechanism of action.

67-84. (canceled)

85. A method for determining whether a combination of plural proteins together form a protein marker for a condition, disease or infection wherein the proteins individually are not markers with a desired level of statistical significance, comprising;
determining proteins which are at altered levels in biological samples from an individual having a condition, disease or infection and control biological samples from an individual not having the condition, disease or infection, which proteins individually have less than the desired level for statistically significant markers by themselves,
selecting two or more of said proteins,
combining the values for two or more of said proteins and determining whether the combination of values is altered in a statistically significant manner,
wherein said combination of proteins results in said protein marker having the desired level of statistically significant differences between biological samples from an individual having a condition, disease or infection and control biological samples from an individual not having the condition, disease or infection.

86. The method of claim 85 wherein the individual has a disease state.

87. The method of claim 85 wherein the proteins are lower molecular weight polypeptides.

88. The method of claim 85 wherein the proteins are detected by mass-spectrometry.

89. The method of claim 85 wherein the individual is an animal.

90. The method of claim 85 wherein the biological sample is from cells or fluids originating from an organism.

91. The method of claim 85 wherein the biological sample is serum.

92. The method according to claim 85 further comprising,
measuring levels of individual proteins in the total proteome of each biological sample, and
comparing these levels of proteins of the total proteome from one type of biological sample to another type.

93. The method of claim 85 wherein the quantity of each protein is measured.

94. A method for determining whether an individual has a normal or abnormal condition, disease or infection comprising;
taking a biological sample from an individual,
measuring levels of each protein in a combination of proteins in the biological sample, wherein the combination of proteins are at altered levels in biological samples from an individual having the particular condition, disease or infection from those in control biological samples from an individual not having the condition, disease or infection, which combination of proteins results in a protein pattern for the presence or absence of the condition, disease or infection,
comparing a pattern formed by the levels of the combination of proteins to known protein patterns for the presence or absence of a particular condition, disease or infection, and
determining the presence or absence of the condition, disease or infection by whether the measured combination of proteins matches the pattern.

95. The method of claim 94 wherein the individual has the disease.

96. The method of claim 94 wherein the individual does not have the condition, disease or infection.

97. The method of claim 94 wherein the proteins are lower molecular weight polypeptides.

98. The method of claim 94 wherein the proteins are detected by mass-spectrometry.

99. The method of claim 94 wherein the individual is an animal.

100. The method of claim 94 wherein the biological sample is from cells or fluids originating from an organism.

101. The method of claim 94 wherein the biological sample is serum.

102. The method of claim 94 wherein the quantity of each protein in the combination of proteins is measured.
